(19) United States
(12) Patent Application Publication
(10) Pub. No.: US 2025/0041361 A1
(43) Pub. Date: Feb. 6, 2025

(54) PROBIOTIC COMPOSITIONS FOR ALLEVIATING GASTROINTESTINAL SYMPTOMS IN SUBJECTS WITH A NEUROLOGICAL DISORDER

(71) Applicant: BIOHM Health Inc., Cleveland, OH (US)

(72) Inventors: Afif Ghannoum, Moreland Hills, OH (US); Michael Haritakis, Hudson, OH (US); Robert Fernald, Lakewood, OH (US)

(21) Appl. No.: 18/570,718

(22) PCT Filed: Jun. 17, 2022
(86) PCT No.: PCT/US2022/034037

§ 371 Date: Dec. 15, 2023

Related U.S. Application Data
(60) Provisional application No. 63/211,967, filed on Jun. 17, 2021.

(51) Int. Cl. A61K 35/747; A61K 9/19; A61K 35/00; A61K 35/745; A61P 31/04

(52) U.S. Cl. CPC A61K 35/747; A61K 9/19; A61K 35/745; A61P 31/04; A61K 2035/115

(57) ABSTRACT

The present invention relates generally to methods and probiotic compositions for alleviating gastrointestinal symptoms in a subject diagnosed with or showing one or more symptoms associated with a neurological disorder (for example, autism spectrum disorder).

### CROSS-REFERENCE TO RELATED APPLICATION

The present application is a national stage entry of PCT application PCT/2022/034037, filed on Jun. 17, 2022, published as WO2022/266469, which in turn, claims the benefit of and priority to U.S. Provisional Patent Application No. 63/211,967, filed Jun. 17, 2021, the entire content of these applications are hereby incorporated by reference in their entirety for all purposes.

### FIELD OF THE INVENTION

The present invention relates generally to compositions and methods for alleviating gastrointestinal symptoms in a subject diagnosed with or having symptoms associated with a neurological disorder (for example, autism spectrum disorder).

### BACKGROUND

Biofilms are formed when unicellular microorganisms live together and form a community that is protected by an exopolysaccharide (EPS) matrix. This EPS matrix typically is a conglomeration of proteins, polysaccharides and extracellular DNA. Biofilm-associated microorganisms differ from their planktonic (freely suspended) counterparts. It is believed that biofilm-forming cells co-aggregate with each other to form coordinated groups attached to a biotic or abiotic surface, the cells are surrounded by a protective EPS matrix, communicate effectively through quorum sensing, and have low metabolic activity that limits the impact of conventional antimicrobials (both antifungal and anti-bacterial agents) acting against actively metabolizing cells or cells in stationary phase.

Microorganisms, including bacteria, fungi, and archaea form biofilms. For a given subject (e.g., a human), many microorganisms (termed microbiota) exist on or within various regions of said subject, e.g., the gut. There is an inextricable link between a host subject's gastrointestinal microbiota and digestion, immunity, and metabolism. Gastrointestinal microbiota are important for vitamin and metabolites biosynthesis and digestion of complex macromolecules such as polysaccharides. Gastrointestinal microbiota, under normal conditions, aid in prevention of colonization of pathogenic microorganisms and in maintaining the integrity and function of the intestinal barrier, and supporting the immune system.

Gastrointestinal disturbances (for example, abdominal pain, diarrhea, and bloating) and metabolic disorders, which are typical of microbial dysbiosis are frequently described in infants with autism spectrum disorders. (See De Angelis M et al., PLoS One, 2013 Oct. 9; 8(10):e76993).

Despite the advances made to date, there is a need for improved compositions and methods for alleviating gastrointestinal symptoms in subjects diagnosed with or showing symptoms associated with neurological disorders.

### SUMMARY OF THE INVENTION

In one aspect, the present invention provides a composition comprising a freeze dried or spray dried blend comprising at least two (e.g., two, three, four, five, or six) isolated and viable non-pathogenic bacterial strains selected from the group consisting of Lactobacillus casei, Bifidobacterium longum, Lactobacillus paracasei, Lactobacillus salivarius, Lactobacillus delbrueckii, and Bifidobacterium breve.

In another aspect, the present invention provides a method of alleviating one or more gastrointestinal symptoms in a subject diagnosed with or having one or more symptoms associated with a neurological disorder, the method comprising consumption by, or administration to the subject of a composition comprising a freeze dried or spray dried blend comprising at least two (e.g., two, three, four, five, or six) isolated and viable non-pathogenic bacterial strains selected from the group consisting of Lactobacillus casei, Bifidobacterium longum, Lactobacillus paracasei, Lactobacillus salivarius, Lactobacillus delbrueckii, and Bifidobacterium breve, thereby to alleviate the gastrointestinal symptoms in the subject. In certain embodiments, the gastrointestinal symptoms comprise at least one of abdominal pain, diarrhea, irregularity, bloating, constipation, and inflammation.

In another aspect, the present invention provides a method of disrupting biofilm in the gastrointestinal tract of a subject diagnosed with or showing one or more symptoms associated with a neurological disorder, the method comprising consumption by, or administration to the subject of a composition comprising a freeze dried or spray dried blend comprising at least two (e.g., two, three, four, five, or six) isolated and viable non-pathogenic bacterial strains selected from the group consisting of Lactobacillus casei, Bifidobacterium longum, Lactobacillus paracasei, Lactobacillus salivarius, Lactobacillus delbrueckii, and Bifidobacterium breve, thereby to disrupt the biofilm in the gastrointestinal tract of the subject. In certain embodiments, the biofilm comprises a pathogenic bacteria and/or pathogenic fungi. In certain embodiments, the pathogenic bacteria comprises Delftia. In certain embodiments, the pathogenic bacteria is selected from the group consisting of Delftia acidovorans Delftia lacustris, Delftia litopenaei, Delftia deserti, and Delftia Tsuruhatensis. In certain embodiments, the pathogenic fungi is selected from the group consisting of Candida tropicalis, Candida glabrata, Candida albicans, Candida parapsilosis, Candida krusei, Candida kefyr, Candida fabianii, Candida lusitaniae, Candida dubliniensis, Candida auris, and Aspergillus.

In yet another aspect, the present invention provides a method of improving gut physiology in a subject diagnosed with or showing one or more symptoms associated with a neurological disorder, the method comprising consumption by, or administration to the subject of a composition comprising a freeze dried or spray dried blend comprising at least two (e.g., two, three, four, five, or six) isolated and viable non-pathogenic bacterial strains selected from the group consisting of Lactobacillus casei, Bifidobacterium longum, Lactobacillus paracasei, Lactobacillus salivarius, Lactobacillus delbrueckii, and Bifidobacterium breve, thereby to improve gut physiology in the subject.

In another aspect, the present invention provides a method of reducing the amount of Delftia in the gut of a subject diagnosed with or showing one or more symptoms associated with a neurological disorder, the method comprising consumption by, or administration to the subject of a composition comprising a freeze dried or spray dried blend comprising at least two (e.g., two, three, four, five, or six) isolated and viable non-pathogenic bacterial strains selected from the group consisting of Lactobacillus casei, Bifidobacterium longum, Lactobacillus paracasei, Lactobacillus salivarius, Lactobacillus delbrueckii, and Bifidobacterium breve, thereby to reduce the amount of Delftia in the gut of the subject.

In certain embodiments, the non-pathogenic bacterial strains are selected from Lactobacillus casei, Bifidobacterium longum subsp. Infantis, Lactobacillus paracasei, Lactobacillus salivarius, Lactobacillus delbrueckii subsp. Bulgaricus, and Bifidobacterium breve. In certain embodiments, the non-pathogenic bacterial strains are selected from the group consisting of Lactobacillus casei, Bifidobacterium longum, and Bifidobacterium breve. In certain embodiments, the non-pathogenic bacterial strains are selected from the group consisting of Lactobacillus casei, Bifidobacterium longum subsp. Infantis, and Bifidobacterium breve.

In certain embodiments, the composition further comprises an enzyme capable of disrupting a biofilm and/or breaking down fiber in the subject. In certain embodiments, the enzyme is selected from the group consisting of amylase, cellulase, hemicellulase, lysozyme, pectinase, DNase I, Serratia peptidase or Serratiopeptidase, hemicellulase/pectinase complex, β-1,3-glucanase, acid protease, alkaline protease, glucoamylase, endoglucanase, xylanase, α-galactosidase, lipase, lysozyme, protease/peptidase complex, dipeptidyl peptidase IV (DPP-IV), chitosanase, bromelain, papain, kiWi protease actinidi, a plant-derived protease, and phytase. In certain embodiments, the enzyme is an amylase selected from the group consisting of Bacillus stearothermophilus amylase, Bacillus amyloliquefaciens amylase, Bacillus subtilis amylase, Bacillus licheniformi amylase, Aspergillus niger amylase, and Aspergillus oryzae amylase. In certain embodiments, the enzyme is α-galactosidase.

In certain embodiments, the composition is formulated as a granulate, pellet, or a powder.

In certain embodiments, the neurological disorder is selected from Alzheimer's disease, autism spectrum disorder (ASD), attention deficit disorder (ADD), dyslexia, and Parkinson's disease. In certain embodiments, ASD is selected from autistic disorder, Asperger syndrome, Heller's syndrome or childhood disintegrative disorder, Rett syndrome, and an unspecified form of pervasive developmental disorder.

In certain embodiments, the composition is provided with (e.g., combined or infused with) a dietary composition for human consumption. In certain embodiments, the dietary composition is one of a cereal based product, rice cake, soy cake, food bar product, cold formed food bar product, custard, yogurt, shake, protein shake, pudding, gelatin, rice milk, soy milk, mashed fruit product, candy, candy bar, and apple sauce.

In one aspect, the present invention provides a composition for use in alleviating one or more gastrointestinal symptoms in a subject diagnosed with or having one or more symptoms associated with a neurological disorder, the composition comprising a freeze dried or spray dried blend comprising at least two (e.g., two, three, four, five, or six) isolated and viable non-pathogenic bacterial strains selected from the group consisting of Lactobacillus casei, Bifidobacterium longum, Lactobacillus paracasei, Lactobacillus salivarius, Lactobacillus delbrueckii, and Bifidobacterium breve.

In one aspect, the present invention provides a composition for use in disrupting biofilm in the gastrointestinal tract of a subject diagnosed with or showing one or more symptoms associated with a neurological disorder, the composition comprising a freeze dried or spray dried blend comprising at least two (e.g., two, three, four, five, or six) isolated and viable non-pathogenic bacterial strains selected from the group consisting of Lactobacillus casei, Bifidobacterium longum, Lactobacillus paracasei, Lactobacillus salivarius, Lactobacillus delbrueckii, and Bifidobacterium breve.

In another aspect, the present invention provides a composition for use in improving gut physiology in a subject diagnosed with or showing one or more symptoms associated with a neurological disorder, the composition comprising a freeze dried or spray dried blend comprising at least two (e.g., two, three, four, five, or six) isolated and viable non-pathogenic bacterial strains selected from the group consisting of Lactobacillus casei, Bifidobacterium longum, Lactobacillus paracasei, Lactobacillus salivarius, Lactobacillus delbrueckii, and Bifidobacterium breve.

In another aspect, the present invention provides a composition for use in reducing the amount of Delftia in the gut of a subject diagnosed with or showing one or more symptoms associated with a neurological disorder, the composition comprising a freeze dried or spray dried blend comprising at least two (e.g., two, three, four, five, or six) isolated and viable non-pathogenic bacterial strains selected from the group consisting of Lactobacillus casei, Bifidobacterium longum, Lactobacillus paracasei, Lactobacillus salivarius, Lactobacillus delbrueckii, and Bifidobacterium breve.

In certain embodiments, the non-pathogenic bacterial strains are selected from Lactobacillus casei, Bifidobacterium longum subsp. Infantis, Lactobacillus paracasei, Lactobacillus salivarius, Lactobacillus delbrueckii subsp. Bulgaricus, and Bifidobacterium breve. In certain embodiments, the non-pathogenic bacterial strains are selected from the group consisting of Lactobacillus casei, Bifidobacterium longum, and Bifidobacterium breve. In certain embodiments, the non-pathogenic bacterial strains are selected from the group consisting of Lactobacillus casei, Bifidobacterium longum subsp. Infantis, and Bifidobacterium breve.

In certain embodiments, the composition further comprises an enzyme capable of disrupting a biofilm and/or breaking down fiber in a subject. In certain embodiments, the enzyme is selected from the group consisting of amylase, cellulase, hemicellulase, lysozyme, pectinase, DNase I, Serratia peptidase or Serratiopeptidase, hemicellulase/pectinase complex, β-1,3-glucanase, acid protease, alkaline protease, glucoamylase, endoglucanase, xylanase, α-galactosidase, lipase, lysozyme, protease/peptidase complex, dipeptidyl peptidase IV (DPP-IV), chitosanase, bromelain, papain, kiWi protease actinidi, a plant-derived protease, and phytase. In certain embodiments, the enzyme is an amylase selected from the group consisting of Bacillus stearothermophilus amylase, Bacillus amyloliquefaciens amylase, Bacillus subtilis amylase, Bacillus licheniformi amylase, Aspergillus niger amylase, and Aspergillus oryzae amylase. In certain embodiments, the enzyme is α-galactosidase.

In certain embodiments, the composition is formulated as a granulate, pellet, or a powder.

In certain embodiments, the neurological disorder is selected from Alzheimer's disease, autism spectrum disorder (ASD), attention deficit disorder (ADD), dyslexia, and Parkinson's disease. In certain embodiments, ASD is selected from autistic disorder, Asperger syndrome, Heller's syndrome, Rett syndrome, childhood disintegrative disorder, and an unspecified form of pervasive developmental disorder.

In certain embodiments, the composition is provided with (e.g., combined or infused with) a dietary composition for human consumption. In certain embodiments, the dietary composition is one of a cereal based product, rice cake, soy cake, food bar product, cold formed food bar product, custard, yogurt, shake, protein shake, pudding, gelatin, rice milk, soy milk, mashed fruit product, candy, candy bar, and apple sauce.

Other features and advantages of the invention will be apparent from the following detailed description and claims.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the present disclosure. In the following description, various embodiments of the present disclosure are described with reference to the following drawings, in which:

FIG. 1 is a diagnostic plot depicting a combined model in which microbiome features, demographic, diet, and clinical features were identified using LASSO logistic modeling. The left vertical line in the plot shows where the cross validation (CV)-error curve hits its minimum of mean-square-error (MSE). The right vertical lines show us the most regularized models with CV-error within 1 standard deviation of the minimum. A value λ (tuning parameter) was chosen according to 5-fold CV, where λ is from the LASSO feature selection algorithm: \hat{β}=arg min_{β}(Likelihood function(β)+λ*Σₖ₌₁^{p}|βₖ|)

FIG. 2 is graphical plot showing the receiving operating characteristic (ROC) curve of model 3 with C-index=0.983, sensitivity of 91%, and specificity of 100%, when cutoff value of the risk (diagnostic) score was 0.6447.

FIG. 3 is a schematic representation of an exemplary process for making a probiotic composition described herein.

### DETAILED DESCRIPTION OF THE INVENTION

The present invention is based, in part, upon the discovery that a composition or a dosage form comprising certain non-pathogenic bacteria can alleviate gastrointestinal symptoms associated with a neurological disorder (for example, autism spectrum disorder) in a mammalian (for example, human) subject. Also provided herein is the discovery that it is possible to prevent or disrupt biofilms comprising pathogenic bacteria and/or fungi, and optionally archaea and/or protozoa, in a subject by administering to the subject a composition or dosage form comprising at least two (e.g., two, three, four, five, or six) isolated and viable non-pathogenic bacterial strains, which when administered promotes a more balanced microbiome, breaks down fiber, and/or promotes the restoration of the natural microbiome in the subject. Unless the context dictates otherwise, the terms composition and dosage form can be used interchangeably herein, where a dosage form is a composition and vice versa. For example, it is contemplated that the composition consumed by, or administered to, a subject can be consumed or administered as a dosage form, for example, as a unit (for example, a spoonful) of the composition. Similarly, it is understood that a dosage form can, for example, comprise a composition described herein. In certain embodiments, a dosage form can comprise a composition in the form of a powder described herein. In certain embodiments, a dosage form can comprise a composition in the form of a capsule that contains such a composition. In certain embodiments, a dosage form can comprise a composition in the form of a granulate. In certain embodiments, a dosage form can comprise a composition in the form of a pellet.

In one aspect, the invention provides a composition comprising an isolated and viable non-pathogenic bacterial strain. The composition comprises at least 2 (e.g., 3, 4, 5, 6 or more) isolated non-pathogenic bacterial strains that are viable in the region of the subject.

In another aspect, the invention provides a composition comprising (i) an enzyme and (ii) at least two non-pathogenic bacterial strains. For example, the composition comprises: (i) one or more (e.g., 1, 2, 3, 4, 5 or more) enzymes and (ii) two or more (e.g., 2, 3, 4, 5, 6 or more) non-pathogenic bacterial strains that are viable (e.g., with or without replication) in the region of the subject.

In another aspect, the invention provides a composition capable of disrupting a biofilm comprising pathogenic bacteria and/or pathogenic fungi disposed within a preselected region of a subject. The composition comprises (i) one or more (e.g., 1, 2, 3, 4, 5 or more) enzymes and (ii) two or more (e.g., 2, 3, 4, 5, 6 or more) non-pathogenic bacterial strains that are viable (e.g., with or without replication) in the region of the subject. In certain embodiments, the enzyme is capable of disrupting the biofilm comprising pathogenic bacteria and/or pathogenic fungi. In certain embodiments, the enzyme breaks down fiber in the subject. In certain embodiments, the non-pathogenic bacterial strains are capable of disrupting the biofilm comprising pathogenic bacteria and/or pathogenic fungi. In certain embodiments, the non-pathogenic bacterial strains break down fiber in the subject.

In another aspect, the invention provides a dosage form capable of disrupting a biofilm comprising pathogenic bacteria and/or pathogenic fungi disposed within a preselected region of a subject. The dosage form comprises a composition comprising (i) one or more (e.g., 1, 2, 3, 4, 5 or more) enzymes and (ii) two or more (e.g., 2, 3, 4, 5, 6 or more) non-pathogenic bacterial strains that are viable (e.g., with or without replication) in the region of the subject. In certain embodiments, the enzyme is capable of disrupting the biofilm comprising pathogenic bacteria and/or pathogenic fungi. In certain embodiments, the enzyme breaks down fiber in the subject. In certain embodiments, the non-pathogenic bacterial strains are capable of disrupting the biofilm comprising pathogenic bacteria and/or pathogenic fungi. In certain embodiments, the non-pathogenic bacterial strains break down fiber in the subject.

### I. Biofilms

The composition or dosage forms of the invention can be used to disrupt and/or replace a biofilm present at a preselected region of a subject, which can include, for example, the gastrointestinal tract in a subject.

Exemplary microorganisms disposed within the biofilm that may be rendered susceptible to disruption by the composition of the dosage form of the invention include, but are not limited to, species of Delftia, including Delftia acidovorans Delftia lacustris, Delftia litopenaei, Delftia deserti and Delftia Tsuruhatensis, species of the Firmicutes phylum, Ascomycota phylum, and Zygomycota phylum for example, Enterococcus spp., including Enterococcus faecalis, Escherichia spp., including Escherichia coli; Chlamydia spp., including Chlamydia pneumonia and Chlamydia trachomatis, Salmonella spp., including Salmonella typhi and Salmonella typhimurium, Pseudomonas spp., including Pseudomonas aeruginosa and Pseudomonas anaerobius, Staphylococcus spp., including Staphylococcus aureus, Staphylococcus capitus, Staphylococcus epidermidis, Staphylococcus saprophyticus, Listeria spp., including Listeria monocytogenes, Helicobacter spp., including Helicobacter pylori, Campylobacter spp., including Campylobacter jejuni, Yersinia spp., including Yersinia pestis, Vibrio spp., including Vibrio cholera, Haemophilus spp. including Haemophilus aphrophilus and Haemophilus influenza, Mycobacterium spp. including Mycobacterium leprae, Mycobacterium tuberculosis, Burkholderia spp., including Burkholderia cepacia, Mycoplasma spp., including Mycoplasma pneumoniae, Klebsiella spp. including Klebsiella pneumoniae, Enterobacter spp. including Enterobacter cloacae, Candida spp., including Candida albicans, Candida dubliniensis, Candida parapsilosis, Candida tropicalis, Candida parapsilosis, Candida glabrata, Candida krusei, Candida Auris and Aspergillus spp., including Aspergillus clavatus, Aspergillus flavus, Aspergillus terreus, and Aspergillus fumigatus.

### Gastrointestinal Tract Biofilms

In certain embodiments, composition or dosage forms described herein can be used to disrupt a biofilm comprising, for example, pathogenic bacteria and/or fungi within the gastrointestinal (GI) tract (for example, esophagus, stomach, upper intestine, and/or the lower intestine) of a subject. In certain embodiments, the preselected region comprises the duodenum, the jejunum, and/or the ileum of the upper GI tract, whereas in other embodiments, the preselected region comprises the appendix, the proximal colon, and/or the rectum of the lower GI tract. In certain embodiments, the subject is diagnosed with or having one or more symptoms associated with a neurological disorder.

In certain embodiments, a composition or dosage form described herein is used to disrupt a biofilm disposed within the GI tract of a subject with an elevated relative abundance of pathogenic bacterial species such as Delftia in the biofilm disposed within the GI tract of the subject when compared to levels typically found in healthy subjects. In certain embodiments, a composition or dosage form described herein comprises non-pathogenic bacterial strains for disrupting or replacing a biofilm containing one or more Delftia pathogenic bacterial species. In certain embodiments, the pathogenic bacterial species of Delftia comprises Delftia acidovorans Delftia lacustris, Delftia litopenaei, Delftia deserti and Delftia Tsuruhatensis.

The bacteria within the biofilm exist in intimate contact with the fungus but may differ in their specific interactions with the fungus. In certain embodiments, the pathogenic bacteria (e.g., Delftia) may be fused to the fungal cells within the biofilm. Alternatively or in addition, the pathogenic bacteria and pathogenic fungi disposed within the biofilm may form a “digestive plaque,” where the bacteria and fungi are protected from antimicrobial drugs and host's immune system. It is understood that digestive plaque can disrupt the normal or healthy microbiome of the GI tract, and cause or be otherwise associated with a GI disease or disorder (e.g., hyperammonemia, Clostridium difficile colitis, hepatic encephalopathy associated with cirrhosis, inflammatory bowel disease, Crohn's disease, ulcerative colitis and/or irritable bowel disease) in a subject diagnosed with or showing one or more symptoms associated with a neurological disorder.

In certain embodiments, a composition or dosage form described herein can be used to disrupt a biofilm in which abundance of a bacterial species, for example, Delftia, is increased significantly, for example, in biofilms disposed within the GI tract of subjects diagnosed with or showing one or more symptoms associated with a neurological disorder when compared to subjects who are not diagnosed with or are not showing one or more symptoms associated with a neurological disorder. In certain embodiments, the increase in the abundance of Delftia can be positively associated with autism.

### II. Dosage Form and Composition

The invention provides compositions or dosage forms that can alleviate GI symptoms in a subject diagnosed with or having symptoms associated with a neurological disorder.

The invention also provides compositions or dosage forms that can prevent and/or disrupt the biofilm at preselected regions of subjects diagnosed with or having one or more symptoms associated with a neurological disorder. In certain embodiments, prevention and/or disruption is by, for example, degrading the EPS matrix and permitting non-pathogenic organisms (e.g., non-pathogenic bacteria and/or fungi) to replace pathogenic organisms present at that region.

The invention also provides compositions or dosage forms that can breakdown fiber in a subject diagnosed with or having one or more symptoms associated with a neurological disorder.

The invention also provides compositions or dosage forms that can improve gut physiology in a subject diagnosed with or having one or more symptoms associated with a neurological disorder.

The invention also provides compositions or dosage forms that can reduce the levels of Delftia in the gut of a subject diagnosed with or showing one or more symptoms associated with a neurological disorder.

In certain embodiments, the composition is formulated as a powdered blend of at least two isolated and viable non-pathogenic bacterial strains that is capable of disrupting a biofilm or preventing formation of biofilm comprising pathogenic bacteria and/or fungi. In certain embodiments, the composition is formulated as a powdered blend of at least two isolated and viable non-pathogenic bacterial strains that is capable of breaking down fiber in a subject diagnosed with or having one or more symptoms associated with a neurological disorder. In certain embodiments, the composition is coated with a functional coating (e.g., controlled release coating) or a non-functional coating (e.g., aesthetic coating). In certain embodiments, the powder blend of at least two isolated and viable non-pathogenic bacterial strains is freeze-dried or spray-dried. In certain embodiments, the freeze-dried or spray-dried blend of at least two isolated and viable non-pathogenic bacterial strains is further blended with an enzyme. In certain embodiments, the enzyme is amylase or α-galactosidase. In certain embodiments, the freeze-dried or spray-dried blend of at least two isolated and viable non-pathogenic bacterial strains is further blended with two enzymes. In certain embodiments, the two enzymes are amylase and α-galactosidase.

### (i) Non-Pathogenic Bacteria

The composition or dosage form comprises two or more (e.g., 2, 3, 4, 5, 6, 7, 8, 9, 10, or more) different non-pathogenic bacterial strains capable of being viable or replicating in a preselected region (e.g., gastrointestinal tract) of a subject. In certain embodiments, the subject is diagnosed with or having one or more symptoms associated with a neurological disorder.

In certain embodiments, exemplary bacterial strains to be included in the composition or dosage form of the present invention may comprise bacterial strains of any one or more of the following bacterial species: Agrococcus jenensis, Alistipes indistinctus, Alistipes massiliensis, Alkalibacterium iburiense, Anoxybacillus kestanbolensis, Bacillus cereus, Bacillus clausii, Bacillus Coagulans, Bacteroides coprophilus, Bacteroides eggerthii, Bacteroides ovatus, Bacteroides fragilis, Bacteroides plebeius, Bacteroides uniformis, Bifidobacterium adolescentis, Bifidobacterium animalis, Bifidobacterium bifidum, Bifidobacterium breve, Bifidobacterium catenulatum, Bifidobacterium catenulatum subsp. Kashiwanohense (DSM 21854), Bifidobacterium longum, Bifidobacterium longum (DSM 20219), Bifidobacterium longum subsp. Infantis, Bifidobacterium pseudocatenulatum, Bifidobacterium pseudocatenulatum (DSM 20438), Bifidobacterium pseudolongum, Blautia obeum, Blautia product, Candidatus azobacteroides, Candidatus portiere, Candidatus Portiera, Clostridium celatum, Clostridium hiranonis, Clostridium neonatale, Clostridium perfringens, Clostridium tyrobutyricum, Collinsella aerofaciens, Collinsella stercoris, Coprococcus eutactus, Corynebacterium stationis, Desulfosporosinus meridiei, Desulfovibrio D168, Dorea formicigenerans, Eggerthella lenta, Erwinia oleae, Faecalibacterium prausnitzii, Lactobacillus acidophilus, Lactobacillus agilis, Lactobacillus casei, Lactobacillus delbrueckii, Lactobacillus delbrueckii subsp. Bulgaricus, Lactobacillus gasseri, Lactobacillus paracasei, Lactobacillus plantarum, Lactobacillus plantarum (DSM 264), Lactobacillus reuteri, Lactobacillus rhamnosus, Lactobacillus ruminis, Lactobacillus salivarius, Lactobacillus zeae, Lactococcus lactis, Listeria weihenstephanensis, Paenibacillus mucilaginosus, Parabacteroides distasonis, Pediococcus acidilactici, Pediococcus pentosaceus, Peptostreptococcus anaerobius, Prevotella copri, Prevotella melaninogenica, Prevotella stercorea, Propionibacterium acnes, Pseudoramibacter eubacterium, Roseburia faecis, Rothia dentocariosa, Rothia mucilaginosa, Ruminococcus bromii, Ruminococcus callidus, Ruminococcus flavefaciens, Ruminococcus lavefaciens, Ruminococcus gnavus, Ruminococcus torques, Salinibacillus aidingensis, Staphylococcus sciuri, Streptococcus anginosus, Streptococcus sobrinus, Streptococcus thermophilus, Tissierella soehngenia, Veillonella dispar, and Veillonella parvula.

In certain embodiments, one or more of the bacterial strains listed in TABLE 1 below are included in the composition or dosage form of the present invention.

TABLE 1

| | |
|---|---|
| Lactobacillus casei | Bifidobacterium lactis |
| Bifidobacterium longum | Lactobacillus delbrueckii subsp. lactis |
| subsp. Infantis | |
| Bifidobacterium animalis | Pediococcus acidilactici |
| Lactobacillus paracasei | Lactobacillus plantarum (DSM 264) |
| Lactobacillus salivarius | Lactobacillus plantarum |
| Lactobacillus delbrueckii subsp. | Lactobacillus rhamnosus |
| Bulgaricus | |
| Bifidobacterium breve | Lactobacillus gasseri |
| Bifidobacterium bifidum | Lactococcus lactis |
| Pediococcus pentosaceus | Lactobacillus reuteri |
| Streptococcus thermophilus | Lactobacillus acidophilus |
| Bifidobacterium catenulatum subsp. | Bifidobacterium longum |
| Kashiwanohense (DSM 21854) | |
| Bifidobacterium longum | Bifidobacterium pseudocatenulatum |
| (DSM 20219) | (DSM 20438) |

In certain embodiments, one or more of the bacterial strains listed in TABLE 2 below are included in the composition or dosage form of the present invention.

TABLE 2

Lactobacillus casei
Bifidobacterium longum subsp. Infantis
Lactobacillus paracasei
Lactobacillus salivarius
Lactobacillus delbrueckii subsp. Bulgaricus
Bifidobacterium breve

In certain embodiments, the composition or dosage form comprises at least two different bacterial strains (e.g., strains listed in TABLE 1 or 2) or at least two of Lactobacillus casei, Bifidobacterium longum, Lactobacillus paracasei, Lactobacillus salivarius, Lactobacillus delbrueckii, and Bifidobacterium breve capable of replicating in the region of a subject diagnosed with or showing one or more symptoms associated with a neurological disorder.

In certain embodiments, the non-pathogenic bacterial strains included in the composition or dosage forms of the present invention may comprise a combination of any two of Lactobacillus casei, Bifidobacterium longum, Lactobacillus paracasei, Lactobacillus salivarius, Lactobacillus delbrueckii, and Bifidobacterium breve. In certain embodiments, the non-pathogenic bacterial strains included in the composition or dosage form may comprise a combination of Lactobacillus casei, Bifidobacterium longum subsp. Infantis, and Bifidobacterium breve. In certain embodiments, the non-pathogenic bacterial strains included in the composition or dosage form may comprise a combination of Lactobacillus casei and Bifidobacterium breve.

In certain embodiments, the composition or dosage form comprises non-pathogenic bacterial strains for disrupting a biofilm containing Delftia in the GI tract of a subject diagnosed with or showing one or more symptoms associated with a neurological disorder. For example, the GI tract biofilm may comprise one or more of Delftia species selected from the group consisting of Delftia acidovorans, Delftia lacustris, Delftia litopenaei, Delftia deserti, and Delftia Tsuruhatensis.

In certain embodiments, the composition or dosage form comprises non-pathogenic bacterial strains for reducing levels of Delftia in the gut of a subject diagnosed with or showing one or more symptoms associated with a neurological disorder.

In certain embodiments, the composition or dosage form of the invention comprises from about 1 billion to about 40 billion, e.g., from about 1 billion to about 5 billion, from about 1 billion to about 10 billion, from about 15 billion to about 40 billion, from about 20 billion to about 40 billion, from about 10 billion to about 30 billion, from about 15 billion to about 30 billion, from about 20 billion to about 30 billion colony forming units of the non-pathogenic bacterial strain(s).

### (ii) Enzymes

The composition or dosage form of the invention further comprises an enzyme capable of disrupting the biofilm. In some embodiments, the enzyme preferably digests or otherwise disrupts/breaks down the EPS matrix of the biofilm. In some embodiments, the enzyme breaks down fiber in a subject diagnosed with or having one or more symptoms associated with a neurological disorder.

The enzyme can be selected from amylase, α-galactosidase, cellulase, hemicellulase, lysozyme, pectinase, DNase I, Serratia peptidase, Serratiopeptidase, hemicellulase/pectinase complex, β-1,3-glucanase, acid protease, alkaline protease, glucoamylase, endoglucanase, xylanase, lipase, lysozyme, protease/peptidase complex, dipeptidyl peptidase IV (DPP-IV), chitosanase, bromelain, papain, kiWi protease actinidi, a plant-derived protease, phytase, zymolase and nuclease. The enzyme may be chosen depending upon the type of biofilm and the microorganisms disposed therein. For example, an amylase enzyme may be used to degrade or otherwise disrupt carbohydrate components of the biofilm, and a nuclease such a DNase I may be used for digest or otherwise disrupt DNA in the biofilm.

In certain embodiments, the composition or dosage form comprises one or more (e.g., 1, 2, 3, 4, 5, or more) different enzymes selected from amylase, α-galactosidase, cellulase, hemicellulase, lysozyme, pectinase, DNase I, Serratia peptidase, Serratiopeptidase, hemicellulase/pectinase complex, β-1,3-glucanase, acid protease, alkaline protease, glucoamylase, endoglucanase, xylanase, lipase, lysozyme, protease/peptidase complex, dipeptidyl peptidase IV (DPP-IV), chitosanase, bromelain, papain, kiWi protease actinidi, a plant-derived protease, phytase, zymolase and nuclease.

In certain embodiments, a composition or dosage form described herein above comprises amylase and/or α-galactosidase. In certain embodiments, a composition or dosage form described hereinabove comprises an amylase selected from Bacillus stearothermophilus amylase, Bacillus amyloliquefaciens amylase, Bacillus subtilis amylase, Bacillus licheniformi amylase, Aspergillus niger amylase, and Aspergillus oryzae amylase. In certain embodiments, a composition or dosage form described hereinabove comprises amylase and α-galactosidase.

In certain embodiments, the composition or dosage form comprises an amylase, for example, from about 20 to about 5,000 SKB units of amylase, 100 to about 5,000 SKB units of amylase, from about 200 to about 4,000 SKB units of amylase, from about 300 to about 2,000 SKB units of amylase or from about 400 to about 1,000 SKB units of amylase. An SKB or Sandstedt, Kneen, and Blish unit refers to the amount of amylase to catalyze 1 μmole substrate per minute. In certain embodiments, composition or dosage form comprises a cellulose, for example, and comprises from about 100 to about 300 CU (Cellulase unit) units per unit composition or dosage form, for example, about 200 CU.

In certain embodiments, the composition or dosage form comprises α-galactosidase, for example, from about 5 to about 200 Galactosidase Units (GalU), for example, from about 10 to about 20 GalU, from about 20 to about 30 GalU, from about 30 to about 40 GalU, from about 40 to about 50 GalU, from about 50 to about 60 GalU, from about 60 to about 70 GalU, from about 70 to about 80 GalU, from about 80 to about 90 GalU, from about 90 to about 100 GalU, from about 100 to about 110 GalU, from about 110 to about 120 GalU, from about 120 to about 130 GalU, from about 130 to about 140 GalU, from about 140 to about 150 GalU, from about 150 to about 160 GalU, from about 160 to about 170 GalU, from about 170 to about 180 GalU, from about 180 to about 190 GalU, from about 190 to about 200 GalU of α-galactosidase.

In certain embodiments, the composition or dosage form comprises about 20 to about 5,000 SKB units of amylase (for example, 100 to about 5,000 SKB units of amylase, from about 200 to about 4,000 SKB units of amylase, from about 300 to about 2,000 SKB units of amylase or from about 400 to about 1,000 SKB units of amylase), selected from α-Amylase, an endo-hydrolase that catalyzes the hydrolysis of internal α-1, 4-glycosidic linkages in starch to yield products like glucose and maltose, β-Amylase, an exo-hydrolase enzyme that hydrolyses α-1, 4-glucan linkages to yield successive maltose units, and γ-Amylase, which cleaves α-1, 6-glycosidic linkages, in addition to cleaving the last α-1, 4-glycosidic linkages to yield glucose, or a combination thereof.

(iii) Manufacturing Process

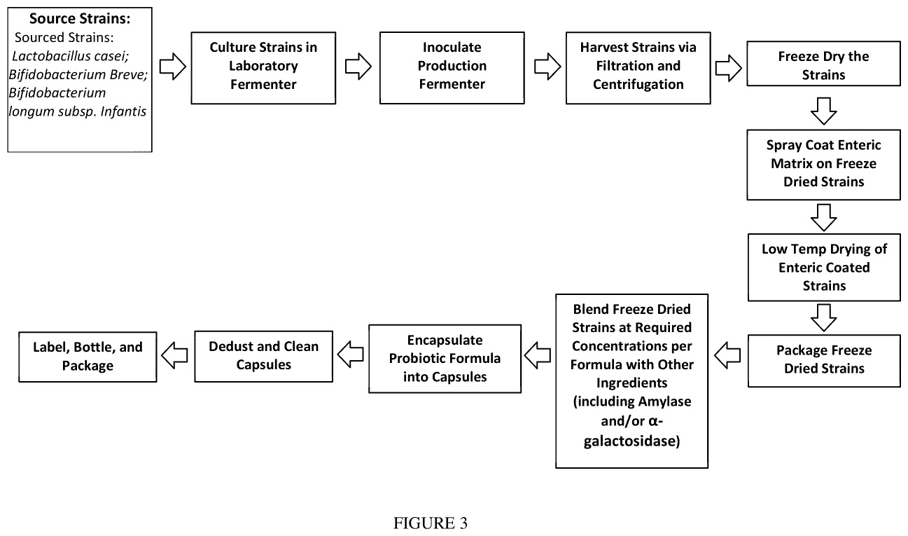

In certain embodiments, a composition of the present invention may be manufactured according to the following process: The non-pathogenic bacterial strains (e.g., Lactobacillus casei, Bifidobacterium longum, and Bifidobacterium breve) are each cultured separately in a small scale fermenter. For each strain, a culture sample is then used to inoculate a corresponding large scale production fermenter. The cultured bacterial strains are harvested via filtration and/or centrifugation, and can then be dried, for example, freeze dried or spray-dried. The dried organisms can then be coated, for example, spray coated with a polymer such as hydroxylpropylmethyl cellulose (HPMC). The coated strains, for example, HPMC-coated strains, can then be subjected to further drying, such as, low temperature drying (e.g., room temperature). The spray coating of freeze-dried bacterial strains is an optional step. The resulting strains (e.g., coated or non-coated) are then blended together at the appropriate concentrations for the specific formulation being manufactured. In order to ensure the active ingredients (e.g., bacterial strains) are at the appropriate concentrations, a manufacturing excipient can also be added. Manufacturing excipients (e.g., sugars, polysaccharides, sugar alcohols, or amino acids) are well-known in the art. Exemplary manufacturing excipients that can be used are leucine, maltodextrin, mannitol and any combination thereof. At this blending stage, if desired, other ingredients specific to the formulation (e.g., dried amylase or dried α-galactosidase) can be added. If desired, the blended composition can then be encapsulated, which can then be packaged into an appropriately labeled container. An exemplary manufacturing process is depicted in FIG. 3.

### (iv) Formulations

The compositions and dosage forms described herein can be used to disrupt a biofilm present in a variety of regions (e.g., the GI tract) within the body of a subject diagnosed with or showing one or more symptoms associated with a neurological disorder. The composition or dosage form may be formulated for use in a variety of delivery systems, for example, oral delivery systems.

In certain embodiments, the composition or dosage form comprises a powder, for example, a coated powder, comprising about 10 million-about 50 billion colony forming units (CFUs) of isolated non-pathogenic bacterial strain(s). In certain embodiments, the composition or dosage form of the present invention comprises about 10 million-about 40 billion, about 10 million-about 30 billion, about 10 million-about 20 billion, about 10 million-about 10 billion, about 10 million-about 5 billion, about 10 million-about 1 billion, about 10 million-about 500 million, about 10 million-about 100 million, about 10 million-about 50 million CFUs of non-pathogenic bacterial strain(s). In certain embodiments, the composition or dosage form of the present invention comprises about 20 million-50 billion, about 50 million-50 billion, about 100 million-about 50 billion, about 200 million-about 50 billion, about 500 million-about 50 billion, about 1 billion-about 50 billion, about 5 billion-about 50 billion, about 10 billion-about 50 billion, about 20 billion-about 50 billion, about 30 billion-about 50 billion, or about 40 billion-about 50 billion CFUs of non-pathogenic bacterial strain(s). In certain embodiments, the composition or dosage form may further comprise an enzyme (e.g., amylase, α-galactosidase, cellulase, hemicellulase, lysozyme, pectinase, DNase I, Serratia peptidase, Serratiopeptidase, hemicellulase/pectinase complex, β-1,3-glucanase, acid protease, alkaline protease, glucoamylase, endoglucanase, xylanase, lipase, lysozyme, protease/peptidase complex, dipeptidyl peptidase IV (DPP-IV), chitosanase, bromelain, papain, kiWi protease actinidi, a plant-derived protease, phytase, zymolase and nuclease).

In certain embodiments, the composition or dosage form comprises about 50 billion, about 40 billion, about 30 billion, about 20 billion, about 10 billion, about 1 billion, about 500 million, about 100 million, about 50 million, or about 10 million CFUs of non-pathogenic bacterial strain(s). In certain embodiments, one capsule comprises about 30 billion colony forming units of non-pathogenic bacterial strain(s) and an enzyme.

### (v) Oral Dosage Form

In certain embodiments, the composition or dosage form may be consumed orally by the subject. In certain embodiments, the composition is consumed as a powder or product containing the powder. In other embodiments, the composition can be in the form of an oral dosage form, for example, where the composition in included, for example, within a capsule, cachet, pill, tablet, lozenge, powder, granule, or as a solution or a suspension in an aqueous or non-aqueous liquid, or as an oil-in-water or water-in-oil liquid emulsion, or as an elixir or syrup, or as pastilles, each containing the requisite number of colony forming units of the non-pathogenic bacteria and non-pathogenic fungus, and optionally the appropriate amount of enzyme.

In certain embodiments, the composition is disposed in a capsule or in a tablet. In certain embodiments, the composition is formulated as a tablet. In certain embodiments, the capsule is a vegetable cellulose capsule. In certain embodiments, the dosage form, for example, capsule or tablet, is coated with a coating, for example, a non-functional aesthetic coating or a functional coating, for example, a controlled release coating. The capsule or tablet may be formulated so as to provide slow or controlled release of the ingredients disposed therein.

In certain embodiments, a dosage form contains a composition comprising (i) an enzyme capable of disrupting the biofilm and (ii) at least two non-pathogenic bacterial strains formulated as a powder and encapsulated in a capsule.

In certain embodiment, the composition or dosage form comprises additives such as calcium carbonate, xylitol, cetyl alcohol, citric acid, natural flavor, monk fruit. In certain embodiments, the composition comprises additives such as cascara sagrada bark, psyllium husk, senna leaf, Flaxseed, aloe vera leaf, licorice root, medium chain triglyceride (MCT) oil. In certain embodiments, the composition or dosage form comprises additives such as a dietary fiber, e.g., inulin (fructooligosaccharides FOS) and apple pectin. In certain embodiments, the composition or dosage form comprises additives such as a blend of spirulina, barley grass, alfalfa leaf, wheat grass, chlorella, dulse, spinach leaf, broccoli, parsley leaf, kale leaf, Echinacea angustifolia root, licorice root, milk thistle seed, Siberian eleuthero root, beet root, rose hips, acai (fruit), green tea leaf, raspberry leaf, blueberry (fruit), goji berry, bilberry (fruit), ashwagandha root, rhodiola root, reishi mushroom, maca root, bee pollen, nettle leaf, Gingko biloba (leaf extract), royal jelly (3× concentrate), grape seed. In certain embodiments, the composition further comprises a vitamin, such as, vitamin C.

### (vi) Coatings

Depending upon the mode of delivery or the region to be treated, the composition or dosage form of the present invention may be prepared as a powdered blend of organisms where the powder can be coated with a non-functional coating (e.g., aesthetic coating) or a functional coating (e.g., controlled release coating to modulate the release of the organisms in, for example, a time- and/or pH-dependent manner). Similarly, the composition or dosage form may be a capsule containing a powdered blend of organisms, where the capsule is coated with a non-functional (e.g., aesthetic coating) or a functional coating (e.g., a controlled release coating to modulate the release of the organisms in, for example, a time- and/or pH-dependent manner). In certain embodiments, controlled release coating is hydroxypropyl methylcellulose (HPMC).

Controlled release coatings can facilitate the continuous release, gradual release, prolonged release, and/or programmed release (e.g., pH-dependent release) of the microorganisms in the compositions or dosage forms disclosed herein.

Exemplary controlled release coatings can be selected from the group consisting of acetate succinate, a polyvinyl derivative (for example, polyvinyl alcohol, polyvinyl acetate, polyvinyl acetate phthalate, a copolymer of vinyl acetate and vinyl pyrrolidone, a copolymer of vinyl acetate and crotonic acid, polyvinylpyrollidone), polyethylene oxide, polyacrylic acid, polysaccharides (for example, modified starch, cross-linked high amylose starch, hydroxypropyl starch, cellulose and cellulose derivatives (for example, microcrystalline cellulose, carboxymethylethyl cellulose, cellulose acetate, methylcellulose, methylhydroxyethylcellulose, ethylcellulose, hydroxypropyl cellulose, hydroxyethylcellulose, hydroxypropylmethyl cellulose, cellulose phthalate, cellulose acetate, cellulose acetate phthalate, cellulose acetate propionate, cellulose-acetate succinate, cellulose acetate butyrate, cellulose-acetate trimellitate, ethylhydroxyethylcellulose, carboxymethylcellulose or methylcarboxymethylcellulose), poloxamer, povidone, alginic acid, sodium alginate, polyethylene glycol, polyethylene glycol alginate, gums (for example, xanthan gum), polymethacrylates (including, for example, a copolymer of methacrylic acid and methyl-methacrylate, and a copolymer of methacrylic acid and ethyl acrylate), a copolymer of methacrylic acid and ethyl acrylate, a copolymer of polymethyl vinyl ether and malonic acid anhydride, a copolymer of polymethyl vinyl ether and malonic acid or the ethyl-, isopropyl-, n-butylesters thereof, zein, and mixtures of the foregoing.

Further examples of controlled release film-coating polymers include, but are not limited to, ethylcellulose (for example, AQUACOAT®, SURELEASE®), methylhydroxypropylcellulose (for example, PHARMACOAT®), acrylic polymers, polyvinylacetates, polyvinyl chlorides, hydroxypropylmethylcellulose acetate succinate (for example, AQOAT), and polyvinyl acetate phthalate (for example, SURETERIC).

In certain embodiments, the coating can be an enteric coating. Enteric coatings can be used to create a barrier that controls the region along the GI tract where the active ingredient(s) are released and absorbed. Enteric coatings may include, for example, a polymer that disintegrates at different rates according to pH. Enteric coatings may include, for example, cellulose acetate phthalate, cellulose acetate trimellitate, methyl acrylate-methacrylic acid copolymers, cellulose acetate succinate, hydroxymethylcellulose, hydroxylpropylmethyl cellulose phthalate, methyl methacrylate-methacrylic acid copolymers, ethylacrylate-methacrylic acid copolymers, methacrylic acid copolymer type C, polyvinyl acetate-phthalate, cellulose acetate phthalate, polyvinyl acetate phthalate, carboxymethylethylcellulose, co-polymerized methacrylic acid/methacrylic acid methyl esters such as, for instance, materials known under the trade name EUDRAGIT® L12.5, L100, or EUDRAGIT® S12.5, S100 or similar compounds used to obtain enteric coatings. Aqueous colloidal polymer dispersions or re-dispersions can be also applied, e.g. EUDRAGIT® L 30D-55, EUDRAGIT® L100-55, EUDRAGIT® S100, EUDRAGIT® preparation 4110D (Rohm Pharma); AQUATERIC®, AQUACOAT® CPD 30 (FMC); KOLLICOAT MAE® 30D and 30DP (BASF); EASTACRYL® 30D (Eastman Chemical).

In certain embodiments, the enteric coating comprises an anionic, cationic, or neutral copolymer based on methacrylic acid, methacrylic/acrylic esters or their derivatives. Cationic polymers often are used for taste-masking and for achieving high bioavailability of active ingredients by their low solubility in oral cavity (pH 5.8-7.4) and high solubility in the stomach (pH 1-3.5), respectively. Anionic polymers have higher water solubility at basic pH than at acidic pH and are used for protecting active ingredients from acid degradation in the stomach and/or enzyme digestion in the intestine.

In certain embodiments, the enteric coating comprises an ethylacrylate-methacrylic acid copolymer. Commercially available enteric coatings include Opadry® AMB, ethylacrylate-methacrylic acid copolymers (e.g., ACRYL-EZE®), dimethylaminoethyl methacrylate-butyl methacrylate-methyl methacrylate copolymer (2:1:1), or poly(methacrylic acid-co-methyl-methacrylate) copolymers, and poly(methacrylic acid-co-methyl-methacrylate) copolymers (e.g., EUDRAGIT®). In certain embodiments, the enteric coating may comprise from about 0.1% to about 10%, from about 1% to about 10%, from about 5% to about 10%, from about 5% to about 20%, from about 8% to about 15%, from about 8% to about 18%, from about 10% to about 12%, or from about 12% to about 16%, of the dosage form (e.g., capsule, tablet or pellet) by weight.

In certain embodiments, the composition or dosage form of the present invention prepared as a powdered mix of two or more isolated and viable non-pathogenic bacterial strains, which may be enteric-coated, or as a capsule containing a powdered mix, where the capsule is enteric-coated, for delivery to the upper intestine. In certain embodiments, the bacterial and fungal strains of the present invention are mixed in a blend to prepare a powder composition, which is then enteric-coated with an cationic copolymer. For example, the powdered mix of the non-pathogenic bacterial and the non-pathogenic fungal strains of the present invention may be coated with a cationic polymer comprising dimethylaminoethyl methacrylate, butyl methacrylate, and methyl methacrylate at a ratio of 2:1:1 (EUDRAGIT® E 100); a cationic copolymer based on dimethylaminoethyl methacrylate, butyl methacrylate, and methyl methacrylate at a ratio of 2:1:1 (EUDRAGIT® EPO); a cationic copolymer based on dimethylaminoethyl methacrylate, butyl methacrylate, and methyl methacrylate at a ratio of 2:1:1 (EUDRAGIT® E 12, 5), an anionic copolymer based on methacrylic acid and ethyl acrylate (EUDRAGIT® L 100-55 (ACRYL-EZE®)), or an equivalent thereof.

In certain embodiments, the bacterial strains of the present invention are mixed in a blend to prepare a powder composition, which is then enteric-coated with an anionic copolymer. For example, the powder blend of the bacterial strains of the present invention may be coated with an anionic copolymer comprising methacrylic acid and methyl methacrylate (e.g., EUDRAGIT® L 100, EUDRAGIT® S 100, EUDRAGIT® L/S (25/75, 50/50, or 75/25 ratio)), or an anionic copolymer comprising methacrylic acid and ethyl acrylate (EUDRAGIT® L 100-55 (ACRYL-EZE®)). Additional but non-limiting examples of the coatings for delivery in the colon include, EUDRAGIT® L 100-55, EUDRAGIT® L 30 D-55, PlasACRYL® HTP 20, EUDRAGIT® L 12,5, EUDRAGIT® FS 100, EUDRAGIT® FS 30 D, and PlasACRYL® T20.

In certain embodiments, the bacterial strains of the present invention are mixed in a blend to prepare granulates or pellets, which are enteric-coated.

In certain embodiments, the powder blend of the bacterial strains of the present invention may be enteric-coated for time-controlled delivery throughout the entire GI-tract to alleviate the gastrointestinal symptoms. For example, the composition or dosage form may be coated with EUDRAGIT® RL 100, EUDRAGIT® RL PO, EUDRAGIT® RL 30 D, EUDRAGIT® RL 12,5, EUDRAGIT® RS 100, EUDRAGIT® RS PO, EUDRAGIT® RS 30 D, EUDRAGIT® RS 12,5, EUDRAGIT® NE 30 D, EUDRAGIT® NE 40 D, and/or EUDRAGIT® NM 30 D.

An exemplary method of producing an oral dosage form comprises selecting the appropriate non-pathogenic bacterial strains to be combined in the dosage form, growing the organisms in a fermenter, either individually or combination depending upon the growth conditions. Once the appropriate amount of biomass has been created, the cells can be harvested via filtration and/or centrifugation. If one or more of the organisms is grown individually, the organisms then can be combined in the appropriate amounts to give the appropriate composition or dosage form, which can then be dried, for example, via freeze drying or spray drying. Alternatively, the organisms, once harvested, can be dried, for example, via freeze drying or spray drying, and then the appropriate dried biomass of each organism can be combined in the appropriate amounts to give the appropriate composition or dosage form. In certain embodiments, manufacturing excipients (e.g., leucine, maltodextrin, mannitol) can be added to obtain appropriate concentrations. In certain embodiments, the dried biomass is prepared in a powder form, and is coated with a functional coating, for example, a controlled release coating (e.g., hydroxypropylmethyl cellulose). In certain embodiments, the dried biomass is prepared in a powder form without a functional coating. The enzyme can be added to the biomass during one or more of each of the foregoing steps, for example, before or after the biomass has been dried. In certain embodiments, the enzyme can be added to the biomass prepared as a powder, for example, before or after coating the powdered biomass when a coating is desired. The resulting biomass (e.g., coated powder) can then be packaged into capsules and packaged into a container.

### III. Probiotic Supplemented Food or Nutritional Compositions

In addition, the invention provides probiotic compositions that can be combined with (e.g., combined with or infused into) in a dietary composition. Exemplary dietary compositions may include but are not limited to: cereal based product, rice cake, soy cake, food bar product, cold formed food bar product, custard, yogurt, shake, protein shake, pudding, gelatin, rice milk, soy milk, mashed fruit product, candy, candy bar, and apple sauce.

In certain embodiments, the dietary probiotic composition (e.g., probiotic supplemented food or nutritional composition) comprises food or nutritional composition (e.g., dietary supplement) combined or infused with (i) two or more (e.g., 2, 3, 4, 5, 6 or more) non-pathogenic bacterial strains and (ii) at least one enzyme.

In certain embodiments, the non-pathogenic bacteria included in the probiotic-infused composition may include Lactobacillus casei, Bifidobacterium longum, Lactobacillus paracasei, Lactobacillus salivarius, Lactobacillus delbrueckii, and Bifidobacterium breve.

In certain embodiments, the enzyme included in the dietary probiotic composition may include amylase, α-galactosidase, cellulase, hemicellulase, lysozyme, pectinase, DNase I, Serratia peptidase or Serratiopeptidase, hemicellulase/pectinase complex, β-1,3-glucanase, acid protease, alkaline protease, glucoamylase, endoglucanase, xylanase, lipase, lysozyme, protease/peptidase complex, dipeptidyl peptidase IV (DPP-IV), chitosanase, bromelain, papain, kiWi protease actinidi, a plant-derived protease, and phytase.

In a specific embodiment, the dietary probiotic composition contains a probiotic composition comprising Lactobacillus casei, Bifidobacterium longum subsp. Infantis, and Bifidobacterium breve. In a specific embodiment, the dietary probiotic composition contains a probiotic composition comprising Lactobacillus casei, Bifidobacterium longum subsp. Infantis, and Bifidobacterium breve and amylase. In a specific embodiment, the dietary probiotic composition contains a probiotic composition comprising Lactobacillus casei, Bifidobacterium longum subsp. Infantis, and Bifidobacterium breve and α-galactosidase. In a specific embodiment, the dietary probiotic composition contains a probiotic composition comprising Lactobacillus casei, Bifidobacterium longum subsp. Infantis, and Bifidobacterium breve amylase and α-galactosidase.

### IV. Beverage Products

The invention also provides beverage products that contain any of the probiotic compositions of the invention described herein. Exemplary beverage products may include but are not limited to: juices, protein shakes, protein smoothies, nutritional drinks, and sports drinks.

In certain embodiments, the beverage product comprises a probiotic composition comprising two or more (e.g., 2, 3, 4, 5 or more) non-pathogenic bacterial strains. In certain embodiments, the beverage product comprises a probiotic composition comprising (i) two or more (e.g., 2, 3, 4, 5 or more) non-pathogenic bacterial strains and (ii) an enzyme.

In certain embodiments, the non-pathogenic bacteria included in the probiotic composition contained in the beverage products may include Lactobacillus casei, Bifidobacterium longum, Lactobacillus paracasei, Lactobacillus salivarius, Lactobacillus delbrueckii, and Bifidobacterium breve.

In certain embodiments, the enzyme included in the probiotic composition contained in the beverage products may include amylase, α-galactosidase, cellulase, hemicellulase, lysozyme, pectinase, DNase I, Serratia peptidase or Serratiopeptidase, hemicellulase/pectinase complex, 0-1,3-glucanase, acid protease, alkaline protease, glucoamylase, endoglucanase, xylanase, lipase, lysozyme, protease/peptidase complex, dipeptidyl peptidase IV (DPP-IV), chitosanase, bromelain, papain, kiWi protease actinidi, a plant-derived protease, and phytase. Such an enzyme is particularly useful for disrupting a biofilm, and the beverage product comprising the enzyme is suitable for consumption by a subject who has a biofilm of pathogenic bacteria and/or fungi.

In a specific embodiment, the beverage product contains a probiotic composition comprising Lactobacillus casei, Bifidobacterium longum subsp. Infantis, and Bifidobacterium breve. In a specific embodiment, the beverage product contains a probiotic composition comprising Lactobacillus casei, Bifidobacterium longum subsp. Infantis, and Bifidobacterium breve and amylase. In a specific embodiment, the beverage product contains a probiotic composition comprising Lactobacillus casei, Bifidobacterium longum subsp. Infantis, and Bifidobacterium breve and u-galactosidase. In a specific embodiment, the beverage product contains a probiotic composition comprising Lactobacillus casei, Bifidobacterium longum subsp. Infantis, and Bifidobacterium breve amylase and α-galactosidase.

### V. Dose

The size of the dose delivered or consumed, will depend upon, among other things, the size and age of the subject, the indication or condition to be treated, and the mode of delivery of the composition or dosage form. In certain embodiments, for example, oral composition or dosage forms may comprise from about 1 mg to about 2,500 mg or 50 mg to about 2,500 mg of the active ingredients (for example, the non-pathogenic bacteria and/or the enzyme). In certain embodiments, the composition or dosage form may comprise from about 1 mg to about 2400 mg, about 1 mg to about 2000 mg, about 1 mg to about 1800 mg, about 1 mg to about 1500 mg, about 1 mg to about 1000 mg, about 1 mg to about 500 mg, about 1 mg to about 400 mg, about 1 mg to about 300 mg, about 1 mg to about 200 mg, about 1 mg to about 100 mg, about 1 mg to about 500 mg, about 10 mg to about 2500 mg, about 20 mg to about 2500 mg, about 30 mg to about 2500 mg, about 40 mg to about 2500 mg, about 50 mg to about 2500 mg, about 100 mg to about 2500 mg, about 200 mg to about 2500 mg, about 300 mg to about 2500 mg, about 400 mg to about 2500 mg, about 500 mg to about 2500 mg, about 600 mg to about 2500 mg, about 700 mg to about 2500 mg, about 800 mg to about 2500 mg, or about 900 mg to about 2500 mg, about 1000 mg to about 2500 mg, about 1200 mg to about 2500 mg, about 1500 mg to about 2500 mg, about 2000 mg to about 2500 mg, about 2200 mg to about 2500 mg, about 2400 mg to about 2500 mg, of the active ingredients (for example, the non-pathogenic bacteria and/or the enzyme).

In certain embodiments, the composition or dosage form may comprise from about 100 mg to about 200 mg, from about 200 mg to about 300 mg, from about 300 mg to about 400 mg, from about 400 mg to about 500 mg, from about 500 mg to about 600 mg, from about 600 mg to about 700 mg, from about 700 mg to about 800 mg, from about 800 to about 900 mg, or from about 900 to about 1000 mg, or from about 1000 to about 1100 mg, or from about 1100 to about 1200 mg, or from about 1200 to about 1300 mg, or from about 1300 to about 1400 mg, or from about 1400 to about 1500 mg, or from about 1500 to about 1600 mg, or from about 1600 to about 1700 mg, or from about 1700 to about 1800 mg, or from about 1800 to about 1900 mg, or from about 1900 to about 2000 mg, or from about 2000 to about 2100 mg, or from about 2100 to about 2200 mg, or from about 2200 to about 2300 mg, or from about 2300 to about 2400 mg, or from about 2400 to about 2500 mg of the active ingredients. In certain embodiments, the composition or dosage form may comprise about 700 mg of the active ingredients.

In certain embodiments, the composition or dosage form comprises about 50, 40, 30, or 20 billion colony forming units of the non-pathogenic bacteria strains. In certain embodiments, the composition or dosage form comprises about 30 billion colony forming units of the non-pathogenic bacteria strains.

In certain embodiments, the composition or dosage form of the invention comprises from about 10 billion to about 40 billion, e.g., from about 15 billion to about 40 billion, from about 20 billion to about 40 billion, from about 10 billion to about 30 billion, from about 15 billion to about 30 billion, from about 20 billion to about 30 billion colony forming units of the non-pathogenic bacterial strain(s).

The composition or dosage forms (either one or multiple units (for example 2, 3, 4, or 5 units, for example, capsules or tablets) may be administered once, twice or three times a day during the treatment period, for example, until the biofilm of interest has been disrupted, prevented from forming, subject's GI symptoms have been improved, and/or the subject's normal microbiome has been restored which could take, for example, one week, two weeks, one month, two months, or three months and one year. In certain embodiments, one capsule, such as one enteric-coated capsule, that comprises about 30 billion colony forming units of non-pathogenic bacterial strain(s) and an enzyme, is administered once, twice, or three times a day during the treatment period, for example, until the biofilm of interest has been disrupted and/or the subject's normal microbiome has been restored which could take, for example, one week, two weeks, one month, two months, or three months and one year.

### VI. Neurological Disorders

The compositions or dosage form of the present disclosure can be used to alleviate gastrointestinal symptoms in a subject diagnosed with or having one or more symptoms associated with a neurological disorder.

### Alzheimer's Disease

The gut microbiota comprises a complex community of microorganism species that resides in our gastrointestinal ecosystem and whose alterations influence not only various gut disorders but also central nervous system disorders such as Alzheimer's disease (AD). AD, the most common form of dementia, is a neurodegenerative disorder associated with impaired cognition and cerebral accumulation of amyloid-O peptides (AD). Most notably, the microbiota-gut-brain axis is a bidirectional communication system that is not fully understood, but includes neural, immune, endocrine, and metabolic pathways. Studies in germ-free animals and in animals exposed to pathogenic microbial infections, antibiotics, probiotics, or fecal microbiota transplantation suggest a role for the gut microbiota in host cognition or AD-related pathogenesis. The increased permeability of the gut and blood-brain barrier induced by microbiota dysbiosis may mediate or affect AD pathogenesis. (See Jiang C, Li G, Huang P, Liu Z, Zhao B. The Gut Microbiota and Alzheimer's Disease. J Alzheimers Dis. 2017; 58(1):1-15). In certain embodiments, the compositions or dosage forms of the present disclosure alleviate one or more gastrointestinal symptoms in a subject diagnosed with Alzheimer's disease. In certain embodiments, the gastrointestinal symptoms comprise abdominal pain, diarrhea, irregularity, bloating, constipation, and inflammation.

### Autism Spectrum Disorder (ASD)

Autism spectrum disorders (ASD) are a spectrum of psychological conditions characterized by social interaction and communication deficits. Symptoms also include repetitive behavior that appear early in childhood, usually before age 3 years and often are accompanied by abnormalities in cognitive functioning. CDC, MMWR, 58 (SS10: 1-20 (2009)) and American Psychiatric Association, Diagnostic and Statistical Manual of Mental Disorders (4th ed), Washington, D.C. (1994). The prevalence of autism in the United States is approximately 1 in every 110 births (1 in 70 boys). CDC, MMWR, 58 (SS10: 1-20 (2009). ASD encompass a range of behaviorally defined conditions. The forms of ASD, including: autistic disorder, Asperger syndrome, Heller's syndrome, Rett syndrome, and an unspecified form of pervasive developmental disorder. Asperger syndrome is closest to autism in signs and causes. Rett syndrome and Child Disintegrative Disorder have similar symptoms as autism, but their etiology may be unrelated. Volkmar, et al., J. Child Psychol. Psychiatry, 50: 108-15 (2009).

Asperger's syndrome is an autism spectrum disorder characterized by significant difficulties in social interaction, along with restricted and repetitive patterns of behavior and interests. It differs from other autism spectrum disorders by its relative preservation of linguistic and cognitive development.

Heller's syndrome or childhood disintegrative disorder is a rare condition characterized by late onset (>3 years of age) of developmental delays in language, social function, and motor skills. CDD has some similarity to autism, and is sometimes considered a low-functioning form of it, but an apparent period of fairly normal development is often noted before a regression in skills or a series of regressions in skills.

Rett syndrome is a genetic neurodevelopmental disorder of the grey matter of the brain that almost exclusively affects girls. It shares many features of autism. The clinical features include small hands and feet and a deceleration of the rate of head growth (including microcephaly in some). Repetitive hand movements, such as wringing and/or repeatedly putting hands into the mouth, are also noted. Gastrointestinal disorders are highly prevalent in patients with Rett syndrome.

Unspecified form of pervasive developmental disorder also known as “Pervasive Developmental Disorder Not Otherwise Specified” (PDD-NOS) is a pervasive developmental disorder (PDD)/autism spectrum disorder (ASD) in which patients have some characteristics of disorders on the autistic spectrum, but do not fit the diagnostic criteria of any of the other autistic disorders thereon. While PDD-NOS shares similarities with classic autism, it tends to be milder. These patients have difficulties socializing, show repetitive behaviors, and are oversensitive to certain stimuli. In their interaction with others they might struggle to maintain eye contact, appear unemotional, or appear to be unable to speak. They may also have difficulty transitioning from one activity to another.

### Attention Deficit Disorder (ADD)

Attention deficit hyperactivity disorder (ADHD) is a chronic condition of inappropriate levels of inattention and/or hyperactivity-impulsiveness that interferes with the quality of social, academic, or occupational functioning. ADHD is one of the most common neuropsychiatric disorders of childhood, with the majority of cases persisting through adulthood. The estimated prevalence of ADHD in the 18-44-year age group is 3.4% worldwide. A number of studies have noted higher rates of abdominal distention, abdominal pain, overweight, and food allergy in children with ADHD. (See Kaplan B J, et al., J Dev Behav Pediatr. 1987; 8:305-310, Hubel et al., Eat Weight Disord., 2006; 11:139-146, Waring M E et al., Pediatrics. 2008; 122:e1-e6, Jameson N D et al., J Child Neurol. 2016; 31:1282-1289).

### Dyslexia

Dyslexia is a neurodevelopmental disorder with a probable genetic basis, and it is generally agreed that more boys than girls are affected (although the gender ratio is higher in referred samples). The core feature of dyslexia is a problem with word decoding, which in turn impacts spelling performance and the development of reading fluency. Dyslexia is persistent across the lifespan, and adult outcomes are variable; although some young people with dyslexia proceed to a university education, others leave school with minimal qualifications. Most adults with dyslexia complain of slow reading, problems of spelling and difficulties with written expression. In addition, problems with working memory, attention and organization are frequently reported. (See Snowling M J., J Res Spec Educ Needs, 2013; 13(1):7-14).

### Parkinson's Disease

Parkinson disease is a neurodegenerative disorder associated with neuronal loss in the substantia nigra, which causes striatal dopamine deficiency, and intracellular inclusions containing aggregates of α-synuclein. Parkinson disease is associated with many non-motor symptoms, including gastrointestinal symptoms that add to overall disability.

### VII. Methods of Alleviating Gastrointestinal Symptoms

The present disclosure provides methods of alleviating gastrointestinal symptoms in a subject diagnosed with or having one or more symptoms associated with a neurological disorder, the method comprising consumption by, or administration to the subject of a composition disclosed herein, thereby to ameliorate the one or more gastrointestinal symptoms in the subject.

The method comprises administering to the subject one or multiple units (for example, capsules, powder, tablets or dietary compositions) of compositions or dosage forms described herein, thereby to alleviate the gastrointestinal symptoms. In certain embodiments, the subject may be a mammal (e.g., human, a companion animal (e.g., dog, cat, or rabbit), or livestock animal (for example, cow, sheep, pig, goat, horse, donkey, and mule, buffalo, oxen, or camel)).

In certain embodiments, administration of the composition or dosage form to the subject diagnosed with or showing one or more symptoms associated with a neurological disorder alleviates one or more gastrointestinal symptoms in said subject. Gastrointestinal symptoms may include nausea, abdominal pain, bloating, constipation, diarrhea, irregularity, inflammation, functional bowel disorder, irritable bowel syndrome, Crohn's disease, ulcers, heartburn, irregularity, gastric neurosis, diverticulosis, cirrhosis, celiac disease, acute gastritis, dyspepsia, gastralgia, gastric carcinoma, gastric vertigo, enteritis, peptic ulcers, cholera morbus, cholera infantum, gastroenteritis, flatulence, inflammatory bowel disease, acid reflux disease, and ulcerative colitis.

In certain embodiments, the alleviation of gastrointestinal symptoms in a subject diagnosed with or showing one or more symptoms associated with a neurological disorder comprises consumption by, or administering to, the subject a probiotic composition comprising two or more (e.g., 2, 3, 4, 5 or more) non-pathogenic bacterial strains. In certain embodiments, the method comprises consumption by, or administering to, the subject a probiotic composition comprising (i) two or more (e.g., 2, 3, 4, 5 or more) non-pathogenic bacterial strains, and (ii) an enzyme. In certain embodiments, the method comprises consumption by, or administering to, the subject a probiotic composition comprising (i) two or more (e.g., 2, 3, 4, 5 or more) non-pathogenic bacterial strains, and (ii) two enzymes.

In certain embodiments, the composition comprises (i) one or more (e.g., 1, 2, 3, 4, 5 or more) enzymes and (ii) two or more (e.g., 2, 3, 4, 5 or more) non-pathogenic bacterial strains. In certain embodiments, the enzyme is capable of disrupting the biofilm comprising pathogenic bacteria and/or pathogenic fungi. In certain embodiments, the enzyme breaks down fiber in the subject. In certain embodiments, the non-pathogenic bacterial strains are capable of disrupting the biofilm comprising pathogenic bacteria and/or pathogenic fungi. In certain embodiments, the non-pathogenic bacterial strains break down fiber in the subject.

### VIII. Methods of Improving Gut Physiology

The present disclosure provides methods of improving gut physiology in a subject diagnosed with or showing one or more symptoms associated with a neurological disorder, the method comprising consumption by, or administration to the subject of a composition disclosed herein, thereby to improve the gut physiology in the subject.

Depending upon the circumstances, the non-pathogenic bacterial strains in the composition or dosage form can (i) displace the pathogenic bacteria in the biofilm, (ii) interfere with the attachment of the pathogenic bacteria/fungus to a substratum of the biofilm, (iii) displace the pathogenic bacteria/fungus from an extracellular polymeric matrix present in the biofilm, (iv) prevent filamentation of the pathogenic fungus in the biofilm, (v) a combination of any of the foregoing, (vi) inhibit the virulence factors of the pathogenic bacteria and fungi (e.g. germination, adherence, etc.).

Under certain circumstances, administration of the composition or dosage form to the subject maintains the total balance of the subject's gut microbiome (including the subject's mycobiome and/or bacteriome), without causing harmful digestive plaque, thereby supporting the subject's optimal digestive health.

In certain embodiments, improving gut physiology in a subject diagnosed with or showing one or more symptoms associated with a neurological disorder comprises consumption by, or administering to, the subject a probiotic composition comprising two or more (e.g., 3, 4, 5 or more) non-pathogenic bacterial strains. In certain embodiments, the method comprises consumption by, or administering to, the subject a probiotic composition comprising (i) two or more (e.g., 3, 4, 5 or more) non-pathogenic bacterial strains, and (ii) an enzyme. In certain embodiments, the method comprises consumption by, or administering to, the subject a probiotic composition comprising (i) two or more (e.g., 3, 4, 5 or more) non-pathogenic bacterial strains, and (ii) two enzymes.

### IX. Methods of Reducing Levels of Pathogenic Bacteria

The present disclosure provides methods of reducing amounts of Delftia in the gut of a subject diagnosed with or showing one or more symptoms associated with a neurological disorder, the method comprising consumption by, or administration to the subject of a composition disclosed herein, thereby to reduce the amounts of Delftia in the subject.

In certain embodiments, a composition or dosage form described herein can be used to reduce the levels of one or more of Delftia bacterial species, including but not limited to, Delftia acidovorans Delftia lacustris, Delftia litopenaei, Delftia deserti and Delftia Tsuruhatensis in the GI tract of a subject. A subject diagnosed with or showing one or more symptoms associated with a neurological disorder have an abundance of Delftia compared to a healthy subject. Abundance of Delftia species in the GI tract of a subject can be determined by measuring the increased detection of Delftia in a biological sample (e.g., fecal sample) obtained from the subject.

In certain embodiments, a composition or dosage form described herein can be used to reduce Delftia levels in a subject diagnosed with or showing one or more symptoms associated with a neurological disorder when compared to healthy subjects.

In certain embodiments, reducing the amount of Delftia in the gut of a subject diagnosed with or showing one or more symptoms associated with a neurological disorder comprises consumption by, or administering to, the subject a probiotic composition comprising two or more (e.g., 2, 3, 4, 5 or more) non-pathogenic bacterial strains. In certain embodiments, the method comprises consumption by, or administering to, the subject a probiotic composition comprising (i) two or more (e.g., 2, 3, 4, 5 or more) non-pathogenic bacterial strains, and (ii) an enzyme. In certain embodiments, the method comprises consumption by, or administering to, the subject a probiotic composition comprising (i) two or more (e.g., 2, 3, 4, 5 or more) non-pathogenic bacterial strains, and (ii) two enzymes

In certain embodiments, administration of a composition or dosage form described here causes a 5% reduction in the amounts of Delftia (e.g., at least 10%, 20%, 30%, 50%, 75%, 90%, 95% reduction or more).

Delftia is not detected in up to 50% of heathy subjects, however, when detected, the value ranges between 0.001 to 0.017 Operational taxonomic units (OTUs).

### X. Methods of Disrupting Biofilm

The present disclosure provides methods of disrupting biofilm in the gastrointestinal tract of a subject diagnosed with or showing one or more symptoms associated with a neurological disorder, the method comprising consumption by, or administration to the subject a composition disclosed herein, thereby to disrupt the biofilm in the gastrointestinal tract of the subject.

In certain embodiments, a composition or dosage form described herein can be used to disrupt a biofilm in which abundance of a bacterial species, for example, Delftia, is increased significantly, for example, in biofilms disposed within the GI tract of diagnosed with or showing one or more symptoms associated with a neurological disorder when compared to healthy subjects.

In certain embodiments, the disruption of biofilm in a subject comprises consumption by, or administering to, the subject a probiotic composition comprising two or more (e.g., 2, 3, 4, 5 or more) non-pathogenic bacterial strains. In certain embodiments, the method comprises consumption by, or administering to, the subject a probiotic composition comprising (i) two or more (e.g., 2, 3, 4, 5 or more) non-pathogenic bacterial strains, and (ii) an enzyme. In certain embodiments, the method comprises consumption by, or administering to, the subject a probiotic composition comprising (i) two or more (e.g., 2, 3, 4, 5 or more) non-pathogenic bacterial strains, and (ii) two enzymes.

### XI. Method of Predicting Susceptibility to Developing Autism

Numerous studies have postulated a connection between the gut-brain axis in Autism Spectrum Disorder (ASD) (Reichelt K L et al., 2009, Ann Clin Psychiatry, 21:205-211; de Theije C G et al., 2011, EurJ Pharmacol 668 Suppl 1:S70-80; Mayer et al., 2014, Bioessays, 36:933-939; Buie T, 2015, Clin Ther, 37:976-983; Kraneveld et al., 2016, Int Rev Neurobiol, 131:263-287; Luna R A et al., 2016, Curr Dev Disord Rep, 3:75-81; Estes M L et al., 2017, Immunity, 47:816-819; Vasquez A, 2017, Ann N YAcad Sci, 1408:5-6; Vuong H E and Hsiao E Y, 2017, Biol Psychiatry, 81:411-423; Fowlie G et al., 2018, IntJ Mol Sci 19; Hicks et al., 2018, Autism Res, 11:1286-1299., 2018; Israelyan and Margolis, 2018, Pharmacol Res, 132:1-6; Sharon G et al., 2019, Cell, 177:1600-1618 e1617). Although these studies have examined this at the bacteriome level, there is a substantial gap in the knowledge surrounding the fungal (mycobiome) connection.

The present disclosure provides a method of predicting the susceptibility of a subject to develop autism. The susceptibility to develop autism in a subject is based on gut microbiome. The method comprises: (a) quantifying the number or density and/or abundance of organisms of at least one pathogenic bacterial strain present in a tissue or body fluid sample harvested from a test subject; and (b) comparing the number or density of organisms quantified in the sample against a threshold value, where the threshold value is the number or density of corresponding organisms typically present in a healthy subject. In certain embodiments, the pathogenic bacterial strain comprises strains from Delftia and Cyanobacteria. In certain embodiments, if the density of a bacterial strain from Cyanobacteria in a test subject is less than that of the healthy subject, the test subject is susceptible to developing autism. In certain embodiments, if the density of a bacterial strain from Delftia in a test subject is greater than that of the healthy subject, the test subject is susceptible to developing autism.

### XII. Definitions

The following definitions are included for the purpose of understanding the present subject matter and for constructing the appended patent claims.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular composition, that composition can be used in the various embodiments of such compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred.

It should be understood that the order of steps or order for performing certain actions is immaterial so long as the present invention remain operable. Moreover, two or more steps or actions may be conducted simultaneously.

At various places in the present specification, components, or features thereof are disclosed in groups or in ranges. It is specifically intended that the description include each and every individual sub-combination of the members of such groups and ranges. By way of other examples, an integer in the range of 1 to 20 is specifically intended to individually disclose 1, 2, 3, 4, 5, 6, 7, 8, 9, 10, 11, 12, 13, 14, 15, 16, 17, 18, 19, and 20.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

As a general matter, compositions specifying a percentage are by weight unless otherwise specified.

As used herein, the terms, “subject,” “patient,” “subject in need thereof,” and “patient in need thereof” are used interchangeably herein, and refer to a living organism, including animals and humans, showing one or more symptoms associated with a neurological disorder that can be treated by the methods and compositions provided herein. The subject can be a human or non-human animal.

Practice of the invention will be more fully understood from the foregoing examples, which are presented herein for illustrative purposes only, and should not be construed as limiting the invention in any way.

### EXAMPLES

The following Examples are merely illustrative and are not intended to limit the scope or content of the invention in any way.

### Example 1—Features of the Gut Microbiome in Subjects with Autism

In this Example, the ability to predict autism based on gut microbiome was tested using logistic models.

To gain more insight into the role of the gut microbiome in autism, rRNA sequencing of bacteriome and mycobiome on stool samples obtained from children with autism and their non-autistic siblings (n=76 subjects) was performed. These two groups would describe the bacterial and fungal microbiota communities in ASD and non-ASD individuals.

### Statistical Methods

The effects of microbiome data (bacteria and fungi at level of phylum, genus and species) and survey data (demographic, diet, lifestyle) on predicting autism were initially estimated using univariate logistic regression model. (See McCullagh P, Nelder J A; 1998; Monographs on statistics and applied probability 37. Boca Roton: Chapman and Hall CRC) McCullagh and Nelder, 1998). The association between survey demographic and personal data and microbiome data used a T-test for two-group comparisons; Pearson correlation coefficient for two continuous measurements and the association of two categorical variables was examined using Chi-square test. A logistic regression model with LASSO (least absolute shrinkage and selection operator) regularization was used for data dimension reduction, microbiome feature salection, and final model construction (Lim M, Hastie T, 2015, J Comput Graplh Stat; 24:627-654).

Different approaches were used to perform LASSO regularizations. Model 1-microbiome (phylum, genus, species) alone: this approach was used for regularization of important bacteria and fungi (phylum, genus and species) that were predictive of autism selected based on univariate analysis; Model 2—survey data alone: this approach was used for regularization of important demographic, and diet factors that were predictive of autism selected based on univariate analysis; Model 3—microbiome (phylum, genus, species) and survey: this approach was used for regularization of important bacteria, fungi (phylum, genus and species), demographic, and diet factors that were predictive of autism selected based on univariate analysis; Model 4—microbiome (all levels) and survey: this approach was used for regularization of important bacteria, fungi (all taxonomic level), demographic, and diet factors that were predictive selected based on univariate analysis. All tests were two-sided and P-value<0.05 was considered statistically significant.

Demographics for the discovery cohort are shown in TABLE 3. Results of the clinical data survey are also incorporated.

TABLE 3

| Factor | Frequency (n = 76) |
|---|---|
| Age at diagnosis: 0/1/2/3 | 11/24/34/7 |
| Race (A/B/I/W) | 9/2/2/63 |
| GIT up (no/yes) | 58/16 |
| GIT low (no/yes) | 34/42 |
| Breast feeding (no/yes) | 14/62 |
| Antibiotics before age 4 | 7/63 |
| | (6 subjects either do |
| | not take antibiotics |
| | or did not respond, |
| | which happens) |
| Sex (female/male) | 22/54 |
| C section (no/yes) | 54/22 |
| Infection during | 50/15 |
| pregnancy (no/yes) | |
| ppinhibitor4 (1 missing) | 65/10 |
| Antibiotics before 6 months | 50/26 |
| Probiotics (1 missing) | 40/35 |
| Factor | Mean (std) |
| exercise | 5.2 (2.3) |
| Number of antibiotics | 6.8 (7.8) |
| Pfood | 7.2 (4.9) |
| Red meat | 2.6 (2.4) |
| Sweets | 5.5 (4.2) |
| Vegetables | 8.0 (4.9) |
| Fish | 0.7 (0.9) |
| Fruit | 9.7 (6.5) |
| Chicken | 4.6 (3.6) |
| Beans | 2.9 (3.7) |
| Whole grain | 7.6 (4.8) |
| BMI | 19.3 (4.6) |
| Prior bowel movement (hrs) | 3.1 (1.1) |
| Age (year) at data collection: | 11.5 (6.5) |
| mean (STD) | |

TABLE 4 depicts the results from the univariate analysis of microbiome data using logistic regression and displaying the associated P-values.

TABLE 4

| Factors (per percentage of abundance increase if not specified) | Odds Ratio | p Value |
|---|---|---|
| p_Cyanobacteria (per 0.01 percentage of abundance increase) | 0.81 | 0.01 |
| c_Chloroplast (per 0.01 percentage of abundance increase)) | 0.75 | 0.02 |
| s_Prevotella nigrescen (per 0.01 percentage of abundance increase) | 0.70 | 0.06 |
| g_Anaerostipes | 0.31 | 0.06 |
| g_Bacteroides | 0.98 | 0.06 |
| o_Streptophyta (per 0.01 percentage of abundance increase) | 0.78 | 0.07 |
| o_Burkholderiales | 1.97 | 0.07 |
| g_Brevundimonas (per 0.01 percentage of abundance increase) | 9.4 | 0.07 |
| o_Boletales | 0.39 | 0.07 |
| c_Betaproteobacteria | 1.82 | 0.08 |
| c_Chytridiomycetes(per 0.01 percentage of abundance increase) | 0.78 | 0.08 |
| f_Chromatiaceae (per 0.01 percentage of abundance increase) | 4.0 | 0.08 |
| s_Galactomyces_geotrichum | 0.99 | 0.08 |
| s_Coprococcus eutactus | 0.45 | 0.08 |
| g_Geotrichum | 0.77 | 0.08 |
| g_Trabulsiella (per 0.01 percentage of abundance increase) | 1.16 | 0.08 |
| f_Clavicipitaceae | 1.72 | 0.09 |
| f_Trichosporonaceae | 0.96 | 0.09 |
| g_Leptothrix (per 0.01 percentage of abundance increase) | 1.85 | 0.09 |
| f_Bacteroidaceae | 0.97 | 0.09 |
| p_Chytridiomycota (per 0.01 percentage of abundance increase) | 0.88 | 0.09 |
| g_Shewanella (per 0.01 percentage of abundance increase) | 1.14 | 0.09 |
| f_Alcaligenaceae | 2.15 | 0.09 |
| g_Delftia (per 0.01 percentage of abundance increase) | 1.5 | 0.09 |
| g_Azospirillum (per 0.01 percentage of abundance increase) | 0.67 | 0.09 |
| f_Marasmiaceae | 3.45 | 0.10 |
| c_Spirochaetes (per 0.01 percentage of abundance increase) | 0.76 | 0.10 |
| g_Metarhizium | 16.85 | 0.10 |

TABLE 5 lists the odds ratio and p-values for factors from univariate analysis of survey darta. As shown in TABLE 5, sex and upper GI disturbances were statistically associated with autism.

TABLE 5

| Factors | Odds ratio | P- value |
|---|---|---|
| Sex (male VS female) | 8.38 | <0.001 |
| upper gastrointestinal | 4.94 | 0.05 |
| disturbances (Yes VS No) | | |

Several LASSO logistic models were performed to examine the ability to predict autism to identify important bacteria and fungi that were significant in univariate analyses. Based on the LASSO logistic model, the identified bacteria were: p_Cyanobacteria, s_Prevotella nigrescens, g_Anaerostipes, g_Bacteroides, s_Coprococcus eutactus, g_Leptothrix, g_Shewanella, and g_Azospirillum.

While the significant fungi identified by univariate analysis included: s_Galactomyces_geotrichum, p_Chytridiomycota, g_Geotrichum, and g_Metarhizium.

Significant demographic, and diet factors that were predictive of autism selected using LASSO logistic regression in univariate analysis were fish consumption, gender, and upper GI disturbances.

The combination of significant factors from both bacteriome (phylum, genus and species) and mycobiome and survey data selected using LASSO logistic model identified the following bacteria: p_Cyanobacteria, s_Prevotella nigrescens, g_Anaerostipes, g_Bacteroides, g_Leptothrix, g_Shewanella, g_Delftia, and g_Azospirillum.

The combination of significant factors from both bacteriome (phylum, genus and species) and mycobiome and survey data selected using LASSO logistic model identified the following fungus: g_Metarhizium. While fish intake, gender, and upper gastrointestinal disturbances were significantly associated with autism. Interestingly, Metarhizium has been used as a biopesticide and thus identification of this fungus may be a result of food consumption. Finally, a combined model in which microbiome features, demographic, diet and clinical features was identified using LASSO logistic modeling, which generated the diagnostic plot shown in FIG. 1.

The performance (Concordance index (C-index)) was evaluated for each of the proposed models. TABLE 6 compares C-index values for the four proposed models (Model 1, Model 2, Model 3, and Model 4) built from different strategies. Although each model had a relatively high C-index, Model 3 was the most robust of the models, as shown in TABLE 6 and was used in further testing.

TABLE 6

| Models | C-index |
|---|---|
| Model 1: Important bacteria and fungi (at taxonomic | 0.935 |
| level of phylum, genus, species) selected using | |
| screening and LASSO | |
| Model 2: Important demographic, diet factors selected | 0.865 |
| using screening and LASSO | |
| Model 3: Important bacteria and fungi ( at taxonomic | 0.983 |
| level of phylum, genus, species) and demographic, | |
| diet factors selected using screening and LASSO | |
| Model 4: Important bacteria and fungi (at all taxonomic | 0.956 |
| level) and demographic, diet factors selected using | |
| screening and LASSO | |

Multivariable logistic modeling analyzed different factors to see what affected autism, making the analysis stronger by eliminating factors that are not relevant (i.e., it focuses the analysis). Results from multivariable logistic modeling using Model 3 are shown in TABLE 7.

TABLE 7

| | Multivariable model | |
|---|---|---|
| | Odds ratio | |
| Factors | (95% CI) | p-value |
| p_Cyanobacteria (per 0.01 percentage | 0.76 (0.62, 0.94) | 0.013 |
| of abundance increase) | | |
| s_Prevotella nigrescens (per 0.01 | 0.84 (0.64, 1.1) | 0.202 |
| percentage of abundance increase) | | |
| g_Anaerostipes (per percentage of | 0.17 (0.01, 2.53) | 0.197 |
| abundance increase) | | |
| g_Bacteroides (per percentage of | 0.98 (0.93, 1.03) | 0.355 |
| abundance increase) | | |
| g_Leptothrix (per 0.01 percentage | 1.26 (0.8, 1.98) | 0.321 |
| of abundance increase) | | |
| g_Delftia (per 0.01 percentage of | 9.99 (1.33, 75) | 0.025 |
| abundance increase) | | |
| g_Shewanella (per 0.1 percentage | 0.37 (0.03, 4.6) | 0.443 |
| of abundance increase) | | |
| g_Azospirillum (per 0.01 percentage | 0.19 (0.05, 0.82) | 0.026 |
| of abundance increase) | | |
| g_Metarhizium (per percentage of | 0.21 (0.01, 7.25) | 0.386 |
| abundance increase) | | |
| Fish (per serving increase) | 0.29 (0.1, 0.82) | 0.02 |
| Sex (Male vs. Female) | 36.79 (2.89, 467.93) | 0.006 |
| upper gastrointestinal disturbances | 2.89 (0.25, 32.87) | 0.392 |

Controlling the effects of other factors (gender and history of upper gastrointestinal disturbances), the odds of having autism was decreased by about 24% per 0.01 percent increase of p_Cyanobacteria (p=0.013). Similarly, the odds of having autism was increased 9.99 times per 0.01 percent increase of g_Delftia (p=0.025). One other genus, (g_Azospirillum), reached a statistically significant p value (p=0.026), therefore, the odds of having autism was decreased by about 81% per 0.01 percent increase of the genus Azospirillum.

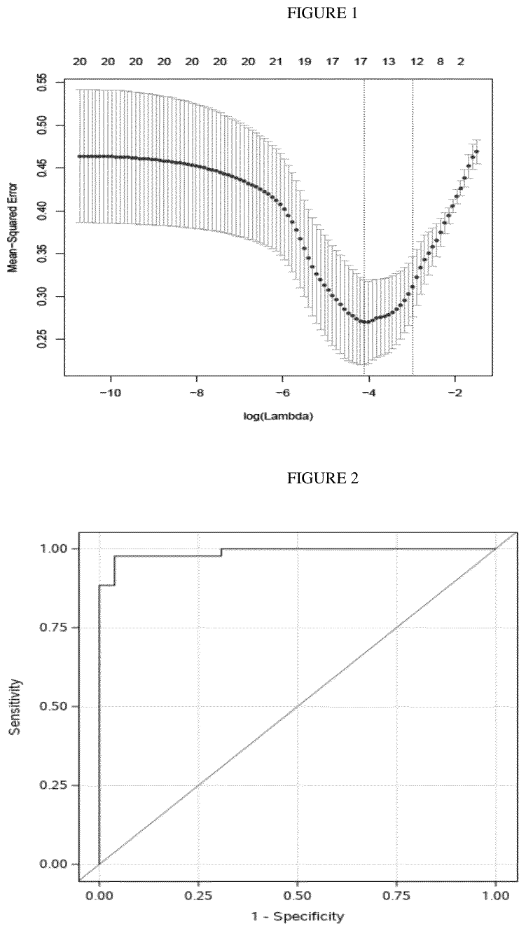

A receiver operating characteristic (ROC) curve analysis shows the final model (model 3) has very good diagnostic performance with C-index of 0.983 and with cutoff value of 0.6447 of the risk score defined above, the sensitivity and specificity for autism diagnosis were 91% and 100%, respectively as shown in FIG. 2.

Findings that autistic patients had a low abundance of p_Cyanobacteria (p=0.013), a bacteria that plays an important role in fiber breakdown, and an increase in the abundance of g_Delftia (p=0.025) a known biofilm producer (Rema T, et al., 2014, Antimicrob Agents Chemother, 58:5673-5686) led to the hypothesis that identifying probiotic strains that can breakdown fiber and inhibit the ability of Delftia to form biofilms will ameliorate the gastrointestinal issues encountered in autistic subjects and boost tolerance to fiber rich diets leading to better quality of life for those living with Autism.

### Example 2—Identification of Probiotic Strains with the Ability to Breakdown Fiber

This example provides a method to identify probiotic strains that are able to breakdown fiber and are capable of modulating the microbiome of subjects with a neurological disorder such as autism.

Numerous non-pathogenic bacterial and fungal strains were tested in this study. TABLE 8 lists different bacterial and fungal strains that were tested.

TABLE 8

| | |
|---|---|
| Bifidobacterium animalis (BSF) | Lactobacillus paracasei (BSF) |
| Bifidobacterium bifidum (BSF) | Lactobacillus plantarum (BSF) |
| Bifidobacterium breve (BIOHM) | Lactobacillus reuteri (BSF) |
| Bifidobacterium lactis (BSF) | Lactobacillus rhamnosus (BIOHM) |
| | lot 200224LR |
| Bifidobacterium longum subps. | Lactobacillus rhamnosus (BIOHM) |
| Infantis (BSF) | lot 200227LR |
| Bifidobacterium longum (BSF) | Lactobacillus salivarius (BSF) |
| Lactobacillus acidophilus (BIOHM) | Lactococcus lactis (BSF) |
| Lactobacillus casei (BSF) | Pediococcus acidilactici (BSF) |
| Lactobacillus delbrueckii subsp. | Pediococcus pentosaceus (BSF) |
| bulgaricus (BSF) | |
| Lactobacillus delbrueckii subsp. | Saccharomyces boulardii (BIOHM) |
| lactis (BSF) | |
| Lactobacillus gasseri (BSF) | Streptococcus thermophilus (BSF) |

### Fiber Fermentation Assay

In order to test the ability of microbial strains to breakdown fibers, a fiber fermentation assay was used.

Media: Two different growth media were used to evaluate the ability of the microbial strains to breakdown fibers: GAM broth (Gifu Anaerobic Media) and Remel Andrade's broth base control (Remel R060102) without carbohydrate.

Test Fibers: four different formulations of Andrade's broth were used as test fibers in this study: (1) 1% Inulin from chicory (Sigma-I2255)-reconstituted 1% Inulin (w/v) in Andrade's base broth without carbohydrate; (2) 1% Agave Inulin (Nuts.com—p73756952)-reconstituted 1% Agave Inulin (w/v) in Andrade's base broth without carbohydrate; (3) 1% Fructooligosaccharides (FOS)—Orafti 95™ (Beneo)-reconstituted 1% FOS (w/v) in Andrade's base broth without carbohydrate; and (4) Control—Andrade's base broth without carbohydrate.

Bacterial Growth: All isolates were grown using GAM for 96 hours in an anaerobic environment using AMG gas (5% CO2, 5% H2, and 90% Nitrogen) at 37° C.

Fiber Metabolism Test: Isolates were diluted to 1×10⁶ cells/ml in Andrade's base broth without carbohydrate and 25 μl were used to inoculate the test fiber solutions.

Evaluation of the ability of strains to breakdown commercially available common fibers: Each fermentable fiber was scored independently by visual inspection and the sum of the score was reported as the Probiotic Fiber Breakdown Score (PFBS), where the largest sum equates to the ability of the strains to ferment 1% Inulin, 1% Agave Inulin, and 1% Fructooligosaccharides (FOS) fiber molecules when challenged as the sole source of carbon. PFBS was based on the following criteria: yellow color=0 (No fiber fermentation); light pink=1 (inefficient fiber fermentation); pink=2 (good fiber fermentation); and red/magenta=3 (very efficient fiber fermentation).

TABLE 9 shows the fermentable fiber scores for 1% Inulin, 1% Agave Inulin, and 1% Fructooligosaccharides (FOS) fiber molecules, as well as the total Probiotic Potential Score (PFBS) for the candidate strains.

TABLE 9

| | Probiotic | | | | |
|---|---|---|---|---|---|
| | Potential | | | | |
| | SCORE | | Agave | | Growth |
| Probiotic Candidates | (PFBS) | Inulin | Inulin | FOS | Rate |
| Lactobacillus casei (BSF) | 9 | 3 | 3 | 3 | Good |
| Bifidobacterium longum subsp. | 9 | 3 | 3 | 3 | Good |
| Infantis (BSF) | | | | | |
| Lactobacillus paracasei (BSF) | 8 | 2 | 3 | 3 | Good |
| Lactobacillus salivarius (BSF) | 8 | 3 | 3 | 2 | Good |
| Lactobacillus delbrueckii subsp. | 8 | 2 | 3 | 3 | Poor |
| Bulgaricus (BSF) | | | | | |
| Bifidobacterium breve | 6 | 0 | 3 | 3 | Good |
| Bifidobacterium bifidum (BSF) | 5 | 0 | 2 | 3 | Good |
| Pediococcus pentosaceus (BSF) | 5 | 0 | 3 | 2 | Good |
| Streptococcus thermophilus (BSF) | 4 | 0 | 3 | 1 | Good |
| Pediococcus acidilactici (BSF) | 4 | 0 | 3 | 1 | Good |
| Lactobacillus plantarum (DSM | 4 | 0 | 2 | 2 | Good |
| 264) | | | | | |
| Bifidobacterium longum (DSM | 3 | 0 | 2 | 1 | Good |
| 20219) | | | | | |
| Lactobacillus plantarum (BSF) | 3 | 0 | 2 | 1 | Good |
| Lactobacillus rhamnosus (BIOHM) | 3 | 0 | 2 | 1 | Good |
| lot 200224LR | | | | | |
| Lactobacillus rhamnosus (BIOHM) | 3 | 0 | 2 | 1 | Good |
| lot 200227LR | | | | | |
| Saccharomyces boulardii (BIOHM) | 3 | 0 | 0 | 3 | Good |
| Lactobacillus gasseri (BSF) | 3 | 0 | 2 | 1 | Good |
| Lactococcus lactis (BSF) | 1 | 0 | 1 | 0 | Good |
| Lactobacillus reuteri (BSF) | 1 | 0 | 1 | 0 | Good |
| Lactobacillus acidophilus | 1 | 0 | 0 | 1 | Good |
| (BIOHM) | | | | | |
| Bifidobacterium longum (BSF) | 1 | 0 | 1 | 0 | Good |
| Bifidobacterium | 0 | 0 | 0 | 0 | Good |
| pseudocatenulatum (DSM 20438) | | | | | |
| Bifidobacterium catenulatum | 0 | 0 | 0 | 0 | Good |
| subsp. Kashiwanohense (DSM | | | | | |
| 21854) | | | | | |
| Komagataella pastoris | 0 | 0 | 0 | 0 | Good |

As shown in TABLE 9, Lactobacillus casei and Bifidobacterium longum subsp. Infantis demonstrated the greatest ability to ferment these fibers. Lactobacillus paracasei, Lactobacillus delbrueckii subsp. Bulgaricus, and Lactobacillus salivarius were also highly efficient at breaking down fiber. Lactobacillus paracasei and Lactobacillus delbrueckii subsp. bulgaricus showed a slight reduction in metabolizing 1% Inulin, while Lactobacillus salivarius showed a slight reduction in metabolizing FOS. TABLE 9 also shows that all candidate probiotic strains demonstrated good growth potential, with the exception of Lactobacillus delbrueckii subsp. Bulgaricus. Bifidobacterium breve was effective in breaking down agave inulin and FOS, although it was not effective in breaking down inulin. Based on this data, six strains with a PFBS of 6 or above (indicated in bold) were selected to evaluate their ability to inhibit biofilm formation by Delftia acidovorans (discussed in EXAMPLE 3).

### Example 3—Identification of Probiotic Strains that Inhibit the Ability of Delftia acidovorans to Produce Biofilms

This example provides a method to identify the ability of probiotic strains selected in the previous EXAMPLE 2 to inhibit the ability of Delftia acidovorans to produce biofilms.

The anaerobic organisms were grown in GAM pre-reduced broth under strict anaerobic conditions. Isolates were incubated for 24-48 hours at 37° C. After incubation, the supernatant from all the strains was filtered through a 0.22 μM filter. Next, the filtrate from each candidate microorganism was combined with GAM broth (1:1) for testing against Delftia biofilms.

### Delftia Biofilm Formation

Sterile 15 mm silicone disks were soaked in fetal bovine serum (FBS) and incubated overnight at 37° C. Delftia acidovorans was grown in GAM pre-reduced media under strict anaerobic conditions. Using a nephelometer, 1×10⁷ cells/ml of Delftia acidovorans were suspended in phosphate buffered saline (PBS). Individual disks were placed in the wells of a 12 well culture plate and 4 ml of Delftia acidovorans cell suspension (1×10⁷ cells/ml) was added. The disks were then incubated at 37° C. for 90 minutes. After 90 minutes, the disks were transferred to single wells in a 24 well plate containing 1.5 ml of the candidate filtrate and GAM broth mix from each candidate probiotic. GAM broth alone was added to a set of disks as positive growth controls. Disks were placed on a rocker and incubated at 37° C. for 96 hours. After 96 hours, each disk was placed in 2 ml of PBS, the biofilm was removed using a cell scraper and cells were suspended. Serial dilutions were made and plated for enumeration of colony forming units (CFUs). All experiments were performed in triplicate. The average log CFUs±SD for each candidate probiotic strain was compared to the positive growth control, p-values of <0.05 were considered significant.

TABLE 10 shows the p-values for the growth of Delftia acidovorans biofilms in the presence of the supernatants of the candidate probiotics when compared to the growth control.

TABLE 10

| | Log CFU of | p-value when |
|---|---|---|
| | Delftia Biofilm | compared to the |
| Delftia Biofilm Inhibition by various organisms | (+/−SD) | growth control |
| Untreated Delftia Control | 6.28 ± 0.02 | |
| Lactobacillus casei (BSF) | 5.17 ± 0.12 | 0.002 |
| Bifidobacterium longum subsp. Infantis (BSF) | 5.09 ± 0.34 | 0.025 |
| Lactobacillus paracasei (BSF) | 5.79 ± 0.13 | 0.017 |
| Lactobacillus salivarius (BSF) | 6.21 ± 0.04 | 0.057 |
| Lactobacillus delbrueckii subsp. Bulgaricus (BSF) | 6.27 ± 0.03 | 0.608 |
| Bifidobacterium breve (BIOHM) | 4.75 ± 0.02 | <0.001 |

As shown in TABLE 10, the supernatant from Lactobacillus casei, Bifidobacterium longum subsp. Infantis, and Lactobacillus paracasei significantly inhibited Delftia acidovorans biofilms (p-values<0.05). Bifidobacterium breve, Bifidobacterium bifidum, and Bifidobacterium longum also significantly inhibited Delftia acidovorans biofilms (p-values<0.05).

Based on the assays described in EXAMPLES 2 and 3, the following strains were selected as the top bacterial strains that are able to both efficiently ferment fibers, inhibit biofilm formation by Delftia acidovorans, and have a good growth rate: Lactobacillus casei, Bifidobacterium longub subsp. Infantis and Bifidobacterium breve. Combinations of these bacterial strains have the ability to provide beneficial effects in the Autism population.

### INCORPORATION BY REFERENCE

The entire disclosure of each of the patent and scientific documents referred to herein is incorporated by reference herein for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

1-38. (canceled)

39. A composition comprising a freeze dried or spray dried blend comprising at least two isolated and viable non-pathogenic bacterial strains selected from the group consisting of Lactobacillus casei, Bifidobacterium longum, Lactobacillus paracasei, Lactobacillus salivarius, Lactobacillus delbrueckii, and Bifidobacterium breve.

40. The composition of claim 39, wherein the composition comprises at least three isolated and viable non-pathogenic bacterial strains selected from the group consisting of Lactobacillus casei, Bifidobacterium longum, Lactobacillus paracasei, Lactobacillus salivarius, Lactobacillus delbrueckii, and Bifidobacterium breve.

41. The composition of claim 39, wherein the composition comprises at least four isolated and viable non-pathogenic bacterial strains selected from the group consisting of Lactobacillus casei, Bifidobacterium longum, Lactobacillus paracasei, Lactobacillus salivarius, Lactobacillus delbrueckii, and Bifidobacterium breve.

42. The composition of claim 39, wherein the non-pathogenic bacterial strains are selected from the group consisting of Lactobacillus casei, Bifidobacterium longum, and Bifidobacterium breve.

43. The composition of claim 39, wherein the non-pathogenic bacterial strains are selected from the group consisting of Lactobacillus casei, Bifidobacterium longum, Lactobacillus paracasei, and Lactobacillus salivarius.

44. The composition of claim 40, wherein the non-pathogenic bacterial strains are selected from the group consisting of Lactobacillus casei, Bifidobacterium longum, Lactobacillus paracasei, and Lactobacillus salivarius.

45. The composition of claim 39, further comprising an enzyme, wherein the enzyme is an amylase selected from the group consisting of Bacillus stearothermophilus amylase, Bacillus amyloliquefaciens amylase, Bacillus subtilis amylase, Bacillus licheniformi amylase, Aspergillus niger amylase, and Aspergillus oryzae amylase.

46. The composition of claim 45, wherein the enzyme is α-galactosidase.

47. The composition of claim 39, wherein the pathogenic fungi is selected from the group consisting of Candida tropicalis, Candida glabrata, Candida albicans, Candida parapsilosis, Candida krusei, Candida kefyr, Candida fabianii, Candida lusitaniae, Candida dubliniensis, Candida auris, and Aspergillus.

48. The composition of claim 39, wherein the composition is active in the gastrointestinal tract of a subject to reduce the presence of a pathogenic bacteria Delftia.

49. The composition of claim 39, wherein the non-pathogenic bacterial strains are selected from the group consisting of Lactobacillus casei, Bifidobacterium longum subsp. Infantis, and Bifidobacterium breve.

50. A method of reducing the amount of Delftia in the gut of a subject diagnosed with or showing one or more symptoms associated with a neurological disorder, the method comprising consumption by, or administration to the subject of a composition comprising a freeze dried or spray dried blend comprising at least two isolated and viable non-pathogenic bacterial strains selected from the group consisting of Lactobacillus casei, Bifidobacterium longum, Lactobacillus paracasei, Lactobacillus salivarius, Lactobacillus delbrueckii, and Bifidobacterium breve;
thereby reducing the amount of Delftia in the gut of the subject.

51. The method of claim 50, wherein the Delftia is in a biofilm.

52. The method of claim 50, wherein the Delftia is selected from the group consisting of Delftia acidovorans Delftia lacustris, Delftia litopenaei, Delftia deserti, and Delftia Tsuruhatensis.

53. The method of claim 51, wherein the biofilm comprises a pathogenic fungi, the pathogenic fungi being selected from the group consisting of Candida tropicalis, Candida glabrata, Candida albicans, Candida parapsilosis, Candida krusei, Candida kefyr, Candida fabianii, Candida lusitaniae, Candida dubliniensis, Candida auris, and Aspergillus.

54. The method of claim 50, wherein the composition comprises at least three isolated and viable non-pathogenic bacterial strains selected from the group consisting of Lactobacillus casei, Bifidobacterium longum, Lactobacillus paracasei, Lactobacillus salivarius, Lactobacillus delbrueckii, and Bifidobacterium breve.

55. The method of claim 50, wherein the composition comprises at least four isolated and viable non-pathogenic bacterial strains selected from the group consisting of Lactobacillus casei, Bifidobacterium longum, Lactobacillus paracasei, Lactobacillus salivarius, Lactobacillus delbrueckii, and Bifidobacterium breve.

56. The method of claim 50, wherein the non-pathogenic bacterial strains are selected from the group consisting of Lactobacillus casei, Bifidobacterium longum, Lactobacillus paracasei, and Lactobacillus salivarius.

57. The method of claim 50, wherein the neurological disorder is selected from Alzheimer's disease, autism spectrum disorder (ASD), attention deficit disorder (ADD), dyslexia, and Parkinson's disease.

58. The composition of claim 39, wherein the composition is combined or infused with a dietary composition for human consumption.
